(19) United States
(12) Patent Application Publication
(10) Pub. No.: US 2026/0174436 A1
(43) Pub. Date: Jun. 25, 2026

(54) CLIP INSTRUMENTS AND CONTROL METHODS THEREOF

(71) Applicant: HANGZHOU AGS MEDTECH CO., LTD., Hangzhou (CN)

(72) Inventors: Chunqi HAN, Hangzhou (CN); Chao YAN, Hangzhou (CN); Yonghua CHENG, Hangzhou (CN); Han YANG, Hangzhou (CN)

(73) Assignee: HANGZHOU AGS MEDTECH CO., LTD., Hangzhou (CN)

(21) Appl. No.: 19/536,482

(22) Filed: Feb. 11, 2026

Related U.S. Application Data
(63) Continuation-in-part of application No. PCT/CN2024/087727, filed on Apr. 15, 2024.

(30) Foreign Application Priority Data
Apr. 10, 2024 (CN) 202410431627.6

(51) Int. Cl. A61B 17/08; A61B 17/10; A61B 17/128

(52) U.S. Cl. CPC A61B 17/083; A61B 17/10; A61B 17/1285

(57) ABSTRACT

Disclosed is a clip instrument and a control method of the clip instrument. The clip instrument includes a sheath, a clip arm, and an accommodating tube. The accommodating tube is releasably connected to the sheath. The clip arm includes at least two clip portions. Proximal ends of the at least two clip portions are movably disposed in the accommodating tube. When the clip arm moves toward a distal direction relative to the accommodating tube, the clip arm opens. When the clip arm moves toward a proximal direction relative to the accommodating tube, the clip arm closes. The accommodating tube is further configured to lock the clip arm.

### CROSS REFERENCE TO RELATED APPLICATIONS

This application is a continuation-in-part of International Application No. PCT/CN2024/087727, filed on Apr. 15, 2024, which claims the priority to Chinese patent application No. 202410431627.6 filed on Apr. 10, 2024, the entire contents of each of which are incorporated herein by reference.

### TECHNICAL FIELD

The present disclosure generally relates to a field of endoscopic medical instruments, and in particular to a clip instrument and a control method of the clip instrument.

### BACKGROUND

An endoscopic hemostatic clip is a medical instrument for hemostasis in an endoscopic surgery. The endoscopic hemostatic clip is usually used in surgical procedures on internal organs such as a gastrointestinal tract and an esophagus to control bleeding and maintain a clear surgical diagram. These clip instruments play a key role in endoscopic surgery, help reduce surgical complications, and improve safety and effectiveness of surgery. The endoscopic hemostatic clip needs to be operated in a relatively narrow space, so a hemostatic clip that is convenient to operate and has strong operational stability is required.

### SUMMARY

One or more embodiments of the present disclosure provide a clip instrument. The clip instrument includes a clip arm and a accommodating tube. A proximal end of the clip arm is provided with a locking portion. The proximal end of the clip arm is movably disposed in the accommodating tube. The accommodating tube includes at least one slot extending along an axial direction. The at least one slot includes a sliding slot and a locking slot. The sliding slot is located at a distal direction of the locking slot. The clip arm switches between an open state and a closed state when the locking portion engages with the sliding slot. The clip arm is in a locked state when the locking portion engages with the locking slot.

One or more embodiments of the present disclosure provides a clip instrument, including a clip arm, a proximal end of the clip arm being provided with a sliding portion; a accommodating tube, the proximal end of the clip arm being movably disposed in the accommodating tube, the accommodating tube including at least one slot extending along an axial direction, and the at least one slot including a sliding slot, a first slot, and a connection slot; the sliding slot being located in a distal direction of the locking slot, and the connection slot being located at a proximal direction of the first slot. The clip instrument further includes a sheath including a connection projection. The connection projection is releasably connected to the connection slot. When the connection portion engages with the sliding slot, the clip instrument switches between an open state and a closed state. when the sliding portion is located in the first slot, the connection projection disengages from the connection slot, and the accommodating tube releases from the sheath.

One or more embodiments of the present disclosure further provide a clip instrument. The clip instrument includes a sheath. The sheath includes an elastic member disposed in a passage of the sheath. The elastic member includes a first connection structure. The clip instrument includes an accommodating tube including a second connection structure. The second connection structure is releasably connected to the first connection structure. The accommodating tube is releasably connected to the sheath. The elastic member is in a stretched state when the first connection structure and the second connection structure are in a limiting engagement. The elastic member drives the first connection structure to retract from the second connection structure when the limiting engagement between the first connection structure and the second connection structure is released.

One or more embodiments of the present disclosure further provide a control method of a clip instrument. The clip instrument includes a clip arm, an accommodating tube, and a sheath. A mandrel is disposed in the sheath. The clip arm includes at least two clip portions. The accommodating tube is releasably connected to the sheath. The clip arm is releasably connected to the mandrel. The control method includes: controlling the clip arm to move from a proximal direction to a distal direction to open the at least two clip portions; controlling the clip arm to move from the distal direction to the proximal direction to close the at least two clip portions; controlling the accommodating tube and the sheath to be released after the at least two clip portions are closed; controlling the clip arm to be locked after the accommodating tube and the sheath are released; and controlling the clip arm and the mandrel to be released after the clip arm is locked.

According to the solutions in the above embodiments, a side wall of the accommodating tube includes a slot. Both the sliding slot and the locking slot are concentrated in the slot, which simplifies an overall structure. The locking portion of the clip arm directly enters the locking slot by moving from the sliding slot toward the proximal direction, which makes a locking operation more convenient. Furthermore, based on the locking effect of the accommodating tube itself on the clip arm, the locking portion is further locked by the locking slot. The locking slot can limit a movement of the locking portion within a certain range. The locking slot limits the clip arm from moving toward the distal direction to prevent the clip portions from opening again. The locking slot limits the clip arm from moving toward the proximal direction to prevent the clip portions from excessively entering the accommodating tube. The locking slot limits the clip arm from moving relative to the accommodating tube in a circumferential direction to prevent a relative rotation between the clip arm and the accommodating tube. The locking reliability between the clip arm and the accommodating tube is improved.

### BRIEF DESCRIPTION OF THE DRAWINGS

The present disclosure is further described in an illustrative manner by way of exemplary embodiments. These exemplary embodiments are described in detail with reference to the accompanying drawings. These embodiments are not limiting. In these embodiments, the same reference numerals denote the same structures, wherein:

FIG. 1A is a schematic structural diagram illustrating an exemplary clip instrument according to some embodiments of the present disclosure;

FIG. 1B is a partial enlarged diagram illustrating a region A of the clip instrument shown in FIG. 1A;

FIG. 2A is a schematic structural sectional diagram illustrating an exemplary clip instrument according to some embodiments of the present disclosure;

FIG. 2B is a schematic structural diagram illustrating an exemplary clip instrument from another perspective according to some embodiments of the present disclosure;

FIG. 2C is a schematic structural diagram illustrating an exemplary accommodating tube of a clip instrument according to some embodiments of the present disclosure;

FIG. 2D is a schematic structural diagram illustrating an exemplary accommodating tube of a clip instrument according to some embodiments of the present disclosure;

FIG. 3A is a schematic sectional structural diagram illustrating an exemplary clip instrument according to some embodiments of the present disclosure;

FIG. 3B is a partial enlarged diagram illustrating a region B of the clip instrument shown in FIG. 3A;

FIG. 4A is a schematic structural diagram illustrating an exemplary accommodating tube according to some embodiments of the present disclosure;

FIG. 4B is a schematic sectional diagram illustrating an exemplary accommodating tube taken along an axial direction according to some embodiments of the present disclosure;

FIG. 5A is a schematic structural diagram illustrating an exemplary clip arm according to some embodiments of the present disclosure, wherein a connection pin and a clip portion are in a disassembled state;

FIG. 5B is a front view illustrating an exemplary clip arm according to some embodiments of the present disclosure;

FIG. 6A is a schematic structural diagram illustrating an exemplary mandrel according to some embodiments of the present disclosure;

FIG. 6B is a schematic structural diagram illustrating an exemplary mandrel according to some embodiments of the present disclosure;

FIG. 7A is a schematic structural diagram illustrating a connection tip and a clip arm of an exemplary clip instrument in a disconnected state according to some embodiments of the present disclosure;

FIG. 7B is a schematic structural diagram illustrating a connection tip and a clip arm of an exemplary clip instrument in a connected state according to some embodiments of the present disclosure;

FIG. 8A is a schematic sectional structural diagram illustrating an exemplary clip instrument according to some embodiments of the present disclosure;

FIG. 8B is a partial enlarged diagram illustrating a region C of the clip instrument shown in FIG. 8A;

FIG. 9 is a schematic structural diagram illustrating an exemplary clip instrument according to some embodiments of the present disclosure;

FIG. 10A is a schematic sectional structural diagram illustrating an exemplary clip instrument according to some embodiments of the present disclosure;

FIG. 10B is a partial enlarged diagram illustrating a region D of the clip instrument shown in FIG. 10A;

FIG. 11A is a schematic structural diagram illustrating an exemplary spring tip according to some embodiments of the present disclosure;

FIG. 11B is a schematic front view illustrating an exemplary spring tip according to some embodiments of the present disclosure;

FIG. 12 is a schematic structural diagram illustrating an exemplary limiting member according to some embodiments of the present disclosure;

FIG. 13A is a schematic structural diagram illustrating an exemplary accommodating tube and an exemplary sheath in an unassembled state according to some embodiments of the present disclosure;

FIG. 13B is a partial enlarged diagram illustrating a region E of the accommodating tube and the sheath according to some embodiments of FIG. 13A;

FIG. 13C is an axial sectional diagram illustrating the accommodating tube and the sheath in the unassembled state according to some embodiments of FIG. 13A.

FIG. 14A is a schematic structural diagram illustrating an exemplary accommodating tube and an exemplary sheath in an assembled state according to some embodiments of the present disclosure;

FIG. 14B is a partial enlarged diagram illustrating a region F of the accommodating tube and the sheath according to some embodiments of FIG. 14A;

FIG. 15 is a schematic structural diagram illustrating an exemplary clip arm and an exemplary accommodating tube according to some embodiments of the present disclosure;

FIG. 16 is a schematic structural diagram illustrating an exemplary clip arm and an exemplary accommodating tube according to other embodiments of the present disclosure;

FIG. 17A is a schematic structural diagram illustrating an exemplary clip instrument in an open state according to some embodiments of the present disclosure;

FIG. 17B is a schematic axial sectional diagram illustrating an exemplary clip instrument in an open state according to some embodiments of the present disclosure;

FIG. 18A is a schematic structural diagram illustrating an exemplary clip instrument in a closed state according to some embodiments of the present disclosure;

FIG. 18B is a schematic axial sectional diagram illustrating an exemplary clip instrument in a closed state according to some embodiments of the present disclosure;

FIG. 19A is a schematic structural diagram illustrating an exemplary accommodating tube and an exemplary sheath in a connected state according to some embodiments of the present disclosure;

FIG. 19B is a partial enlarged diagram illustrating a region G of the clip instrument according to some embodiments of FIG. 19A;

FIG. 19C is a schematic structural diagram illustrating an exemplary accommodating tube and an exemplary sheath in a released state according to some embodiments of the present disclosure;

FIG. 19D is a partial enlarged diagram illustrating a region H of the clip instrument according to some embodiments of FIG. 19C;

FIG. 20A is a schematic structural diagram illustrating an exemplary clip instrument in a locked state according to some embodiments of the present disclosure;

FIG. 20B is a schematic axial sectional diagram illustrating an exemplary clip instrument in a locked state according to some embodiments of the present disclosure;

FIG. 21A is a schematic structural diagram illustrating an exemplary mandrel and an exemplary clip arm in a connected state according to some embodiments of the present disclosure;

FIG. 21B is a schematic axial sectional diagram illustrating an exemplary mandrel and an exemplary clip arm in a connected state according to some embodiments of the present disclosure;

FIG. 21C is a schematic structural diagram illustrating an exemplary mandrel and an exemplary clip arm in a released state according to some embodiments of the present disclosure;

FIG. 21D is a schematic axial sectional diagram illustrating an exemplary mandrel and an exemplary clip arm in a released state according to some embodiments of the present disclosure;

FIG. 22 is a flowchart illustrating an exemplary process for controlling a clip instrument according to some embodiments of the present disclosure.

Reference numerals denote:

10, clip instrument; 100, clip arm; 110, locking portion; 120, clip portion; 121, distal joint portion; 122, bending portion; 123, proximal joint portion; 125, first connection portion; 1251, pin hole; 126, second connection portion; 1261, connection hole; 127, installation slot; 130, connection pin; 140, sliding portion; 150, stop portion; 200, delivery portion; 210, sheath; 211, first connection structure; 2111, connection projection; 212, elastic member; 2121, spring; 2122, spring tip; 2123, first engagement portion; 2124, second engagement portion; 2125, first end face; 2126, second end face; 220, mandrel; 221, connection tip; 222, guide slope; 223, guide slot; 300, control portion; 310, fixed handle; 320, sliding handle; 400, accommodating tube; 410, slot; 411, sliding slot; 412, locking slot; 413, first slot; 4131, third limiting portion; 414, connection slot; 415, second slot; 416, third slot; 420, second connection structure; 430, concession slot; 440, first limiting portion; 450, second limiting portion; 500, limiting member; 510, latching portion; and 520, operation portion.

### DETAILED DESCRIPTION

To more clearly illustrate the technical solutions in the embodiments of the present disclosure, the accompanying drawings used in the description of the embodiments are briefly introduced below. Obviously, the accompanying drawings in the following description are merely some examples or embodiments of the present disclosure. For those skilled in the art, the present disclosure can be applied to other similar scenarios based on these accompanying drawings without creative efforts. Unless obviously obtained from the context or the context illustrates otherwise, the same numeral in the drawings refers to the same structure or operation.

As shown in the present disclosure and the claims, unless the context clearly indicates an exception, the terms “a,” “an,” “one,” and/or “the” are not intended to be singular only and may also include the plural. Generally, the terms “include” and “comprise” only indicate the inclusion of explicitly identified steps and elements. These steps and elements do not constitute an exclusive list. A method or device may also include other steps or elements.

Flowcharts are used in the present disclosure to illustrate operations performed by a system according to embodiments of the present disclosure. It should be understood that preceding or following operations are not necessarily performed precisely in sequence. Conversely, each operation may be processed in reverse order or simultaneously. Meanwhile, other operations may be added to these processes, or one or more operations may be removed from these processes.

A clip instrument is a common surgical instrument applied to an endoscope. During a surgical procedure, the clip instrument functions to stop bleeding by clipping a tissue wound. After a clip portion of a conventional clip instrument clips a tissue, the clip portion is locked by a locking member. Then, the clip portion and the locking member are released together from a sheath and remain at a wound position.

The conventional clip instrument typically has a complex structure. Various problems, such as insecure locking or difficulty in separation during release, are prone to occur, especially during locking and releasing processes of the clip instrument. For example, a locking member of the clip instrument forms a limiting fit with a clip arm to achieve locking. Problems such as insecure locking or complex locking structures exist, leading to a risk of accidental detachment after the clip portion clips tissue. As another example, a connection structure between the sheath and an accommodating tube of the clip instrument is complex, resulting in complicated separation operations or damage to the clip instrument during separation, thereby affecting a clipping effect.

Given this, in some embodiments of the present disclosure, a clip instrument is expected to be provided. The clip instrument includes an accommodating tube and a clip arm. The accommodating tube includes a slot. The clip arm includes a slider. Operations such as opening, closing, locking, and releasing of the clip arm are achieved through sliding of the slider in the slot. The clip instrument has a simple structure and can be simply operated. The clip instrument has advantages such as a secure locking between the accommodating tube and the clip arm and an easy release between the accommodating tube and the sheath.

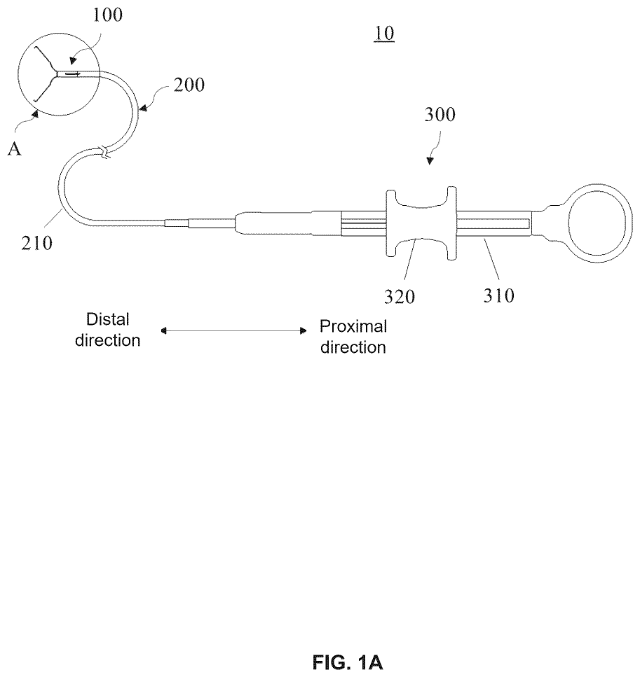

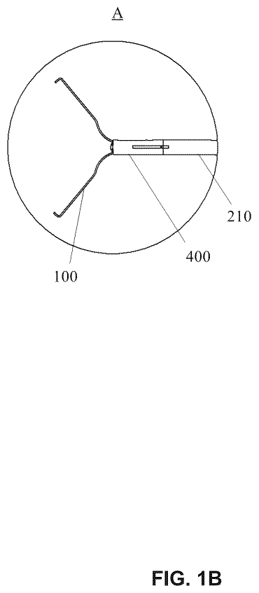

FIG. 1A is a schematic structural diagram illustrating an exemplary clip instrument according to some embodiments of the present disclosure. FIG. 1B is a partial enlarged diagram illustrating a region A of the clip instrument shown in FIG. 1A.

As shown in FIG. 1A and FIG. 1B, in some embodiments, a clip instrument 10 includes a clip arm 100, a delivery portion 200, and a control portion 300. The control portion 300 is disposed at a proximal end of the delivery portion 200. The clip arm 100 is disposed at a distal direction of the delivery portion 200. The terms “proximal end” and “distal end” involved in the embodiments of the present disclosure may indicate directions. The terms refer to an axial direction of the clip instrument 10 (e.g., an extension direction of the sheath 210 of the delivery portion 200 within an endoscopic channel). A side toward an operator is the “proximal end.” A side extending into a human body for treatment is the “distal end.” The terms “proximal end” and “distal end” may also indicate portions of structures located in corresponding directions and should not be understood as indicating only ends.

In some application scenarios, the delivery portion 200 has a good possibility. The delivery portion 200 and the clip arm 100 at the distal direction of the delivery portion 200 enter the human body through an endoscopic operation passage to approach a tissue to be clipped. The tissue refers to an organ tissue of the human body or other organism. The control portion 300 is located outside the human body or other organism. A user controls the clip arm 100 to perform a surgical operation by manipulating the control portion 300. For example, the clip arm 100 clips a wound of the tissue to keep the wound closed, thereby assisting wound healing.

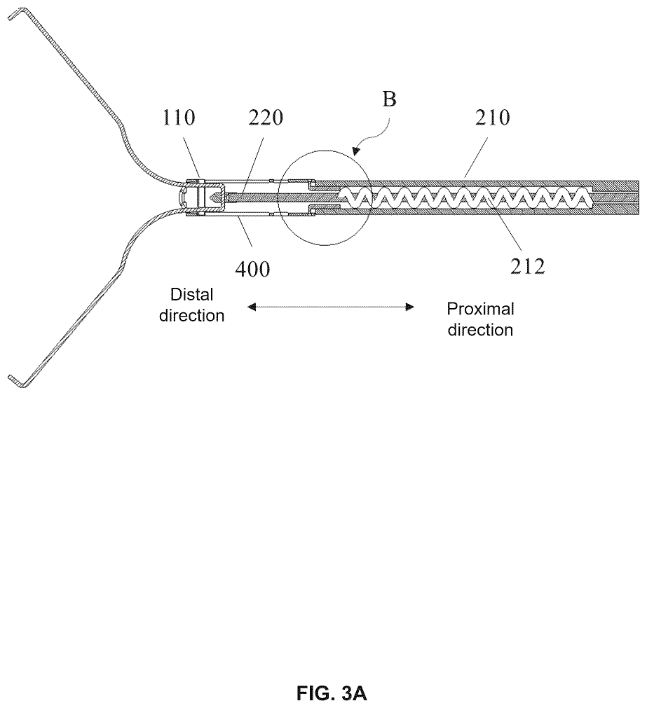

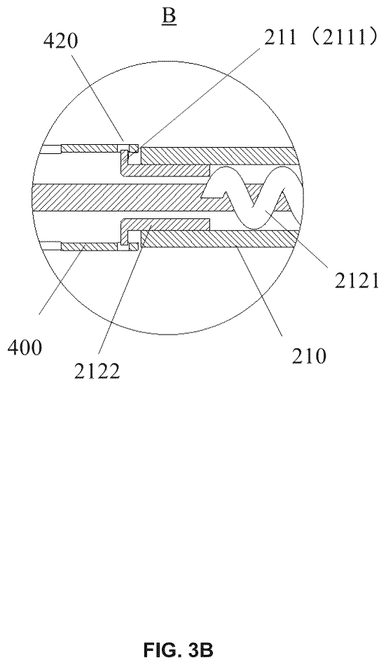

In some embodiments, the delivery portion 200 includes the sheath 210 and a mandrel 220 (the mandrel 220 is shown in FIG. 3A). The mandrel 220 is disposed in the passage of the sheath 210 and extends along an axial direction of the sheath 210. A proximal end of the mandrel 220 is connected to the control portion 300. A distal end of the mandrel 220 is connected to the clip arm 100. The terms “axial direction” and “radial direction” involved in the embodiments of the present disclosure may indicate directions. The “radial direction” is perpendicular to the “axial direction.” Alternatively, the axial direction is an extension direction of the passage of the sheath 210, and the radial direction is perpendicular to the extension direction of the passage of the sheath 210.

In some embodiments, the sheath 210 has flexibility and bends in any direction. In some embodiments, the control portion 300 includes a fixed handle 310 and a sliding handle 320. The sliding handle 320 is slidable relative to the fixed handle 310 in the axial direction. A distal end of the sliding handle 320 is fixedly connected to the proximal end of the mandrel 220. The user controls the sliding handle 320 to move along the axial direction of the fixed handle 310 outside the body to control an axial movement of the mandrel 220 within the passage of the sheath 210, causing the clip arm 100 to complete corresponding surgical operations, such as opening, closing, locking, and releasing.

In some embodiments, the clip instrument 10 further includes an accommodating tube 400. The accommodating tube 400 is releasably disposed at the distal direction of the sheath 210. A proximal end of the clip arm 100 is movably fitted within a passage of the accommodating tube 400 and is releasably connected to the mandrel 220.

The present disclosure provides various embodiments of the clip instrument, aiming to solve problems such as an insecure locking of the clip instrument 10, complex locking structures, and a difficulty in releasing the clip arm 100 and the sheath 210. Provided that there is no contradiction, a certain feature, structure, or feature among various embodiments may be appropriately combined or referenced.

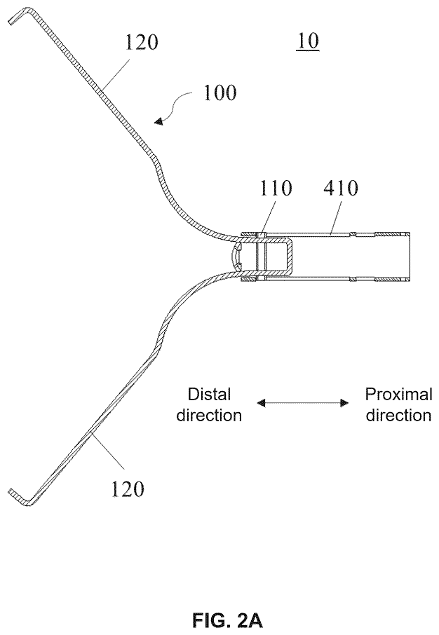

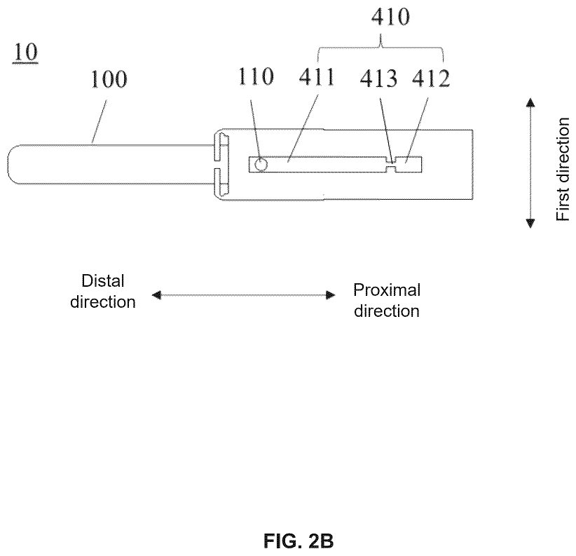

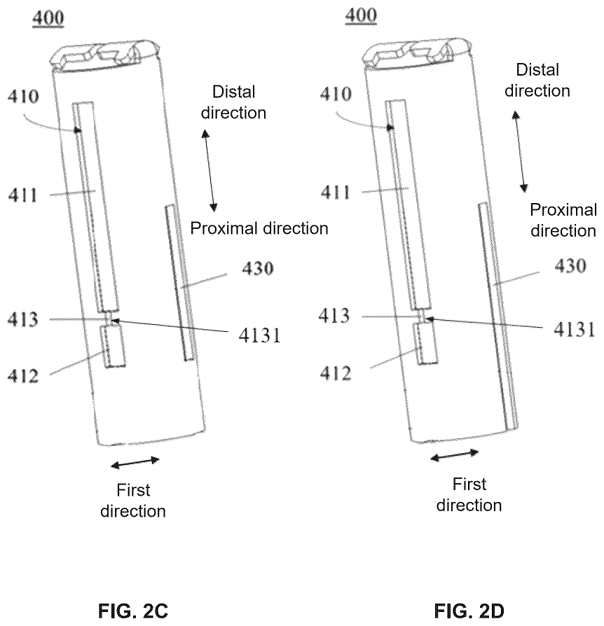

FIG. 2A is a schematic structural sectional diagram illustrating the clip instrument 10 according to some embodiments of the present disclosure. FIG. 2B is a schematic structural diagram illustrating the clip instrument 10 from another perspective according to some embodiments of the present disclosure.

As shown in FIG. 2A and FIG. 2B, Embodiment 1 of the present disclosure provides the clip instrument 10. The clip instrument 10 includes the clip arm 100 and the accommodating tube 400.

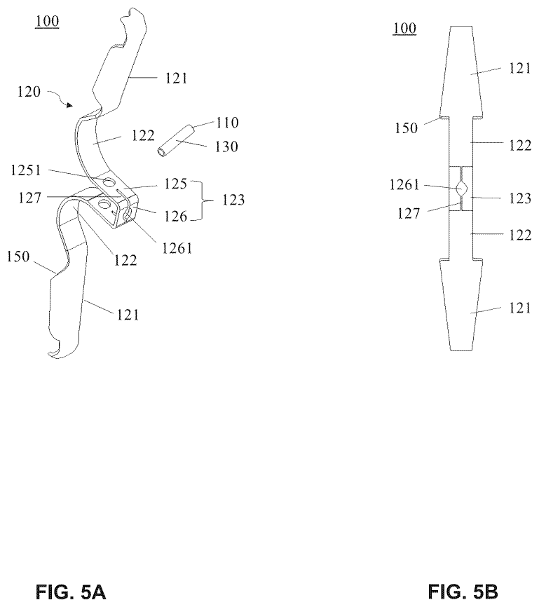

In some embodiments, a proximal end of the clip arm 100 is provided with a locking portion 110. The proximal end of the clip arm 100 is movably disposed in the accommodating tube 400. In some embodiments, the clip arm 100 includes at least two clip portions 120. The at least two clip portions 120 clip a target object (e.g., a tissue wound) through operations such as opening and closing, causing the target object to remain closed. When the clip arm 100 moves from the proximal direction toward a distal direction relative to the accommodating tube 400, so that when distal ends of the at least two clip portions 120 move away from each other, the clip arm 100 is in an open state. When the clip arm 100 moves from the distal direction toward the proximal direction relative to the accommodating tube 400, so that when the distal ends of the at least two clip portions 120 move closer to each other, the clip arm 100 is in a closed state. In some embodiments, the clip arm 100 includes a connection pin 130. Two end portions of the connection pin 130 constitute the locking portion 110. For more details regarding components such as the locking portion 110 and the connection pin 130, please refer to the more specific descriptions below (e.g., the related descriptions in FIG. 5A).

In some embodiments, the accommodating tube 400 includes at least one slot 410 extending along the axial direction. The slot 410 includes a sliding slot 411 and a locking slot 412. The sliding slot 411 is located at a distal direction of the locking slot 412. That is, both the sliding slot 411 and the locking slot 412 are parts of the slot 410. The sliding slot 411 and the locking slot 412 may be arranged sequentially along the axial direction of the slot 410. For example, the locking slot 412 is located at one end of the sliding slot 411 in the axial direction. One end (e.g., the proximal end) of the sliding slot 411 communicates with one end (e.g., the distal end) of the locking slot 412. For example, the sliding slot 411 directly communicates with the locking slot 412 or communicates through a first slot 413 described below. When the locking portion 110 of the clip arm 100 engages with the sliding slot 411, the clip arm 100 switches between the open state and the closed state. When the locking portion 110 of the clip arm 100 engages with the locking slot 412, the clip arm 100 is in a locked state. When the locking portion 110 of the clip arm 100 passes over the first slot 413 and engages with the locking slot 412, the clip arm 100 is in the locked state. The locking slot 412 may limit an axial movement and a circumferential movement of the locking portion 110 within a certain range. For example, the locking slot 412 limits a movement of the clip arm 100 toward the distal direction, which makes the clip portion 120 open. The locking slot 412 may limit the movement of the clip arm 100 toward the proximal direction, which makes the clip portion 120 to excessively enter the accommodating tube 400. The locking slot 412 may limit a rotation of the clip arm 100 relative to the accommodating tube 400. After the locking slot 412 limits a relative movement between the clip arm 100 and the accommodating tube 400 in various directions, the clip arm 100 and the accommodating tube 400 are locked.

According to the solution in the above embodiment, a sidewall of the accommodating tube 400 includes the slot 410. Both the sliding slot 411 and the locking slot 412 are concentrated in the slot 410, thereby simplifying the overall structure. The locking portion 110 of the clip arm 100 directly enters the locking slot 412 by moving from the sliding slot 411 toward the proximal direction, making the locking operation more convenient. Furthermore, based on a locking effect of the accommodating tube 400 itself on the clip arm 100, the locking portion 110 is further locked by the locking slot 412. The locking slot 412 limits the movement of the locking portion 110 within a certain range, limits the movement of the clip arm 100 toward the distal direction to prevent the clip portion 120 from opening again, limits the movement of the clip arm 100 toward the proximal direction to prevent the clip portion 120 from excessively entering the accommodating tube 400, and limits the movement of the clip arm 100 relative to the accommodating tube 400 in a circumferential direction to prevent a relative rotation between the clip arm 100 and the accommodating tube 400. In this way, the reliability of locking between the clip arm 100 and the accommodating tube 400 is improved.

In some embodiments, as shown in FIGS. 2B-2D, the accommodating tube 400 further includes at least one concession slot 430. The concession slot 430 is spaced apart from the slot 410 along the circumferential direction of the accommodating tube 400. The concession slot 430 extends from a middle portion of the accommodating tube 400 to a proximal edge of the accommodating tube 400 (the concession slot 430 may or may not penetrate the proximal edge of the accommodating tube 400). In some embodiments, an extension direction of the concession slot 430 is parallel to an extension direction of the slot 410. In other embodiments, the extension direction of the concession slot 430 forms an angle with the extension direction of the slot 410. For example, the angle is less than 60 degrees. As another example, the angle is less than 45 degrees. As another example, the angle is less than 30 degrees. There are one or more concession slots 430. When there are a plurality of concession slots 430, the plurality of concession slots 430 are spaced apart along the circumferential direction of the accommodating tube 400. By providing the concession slot 430 spaced apart from the slot 410 along the circumferential direction of the accommodating tube 400, a tube body of the accommodating tube 400 (a portion of the tube body between the concession slot 430 and the slot 410) may be deformed, thereby avoiding an excessive resistance to the movement of the locking portion 110 within certain sections of the slot 410 (e.g., the first slot 413).

The tube body of the accommodating tube 400 located between the concession slot 430 and the slot 410 may form a beam structure. In some embodiments, as shown in FIG. 2C, two ends of the concession slot 430 along an axial direction of the concession slot 430 are closed. In this case, the concession slot 430 does not penetrate the proximal edge of the accommodating tube 400. The tube body of the accommodating tube 400 located between the concession slot 430 and the slot 410 forms a simply supported beam structure. A proximal end and a distal end of the tube body of the accommodating tube 400 located between the concession slot 430 and the slot 410 may be respectively regarded as two support points of the simply supported beam structure. Through such a configuration, both ends of the tube body of the accommodating tube 400 located between the concession slot 430 and the slot 410 may be supported, thereby avoiding a significant vibration or a radial warping deformation of the portion of the tube body along the radial direction of the accommodating tube 400, and ensuring the structural stability of the accommodating tube 400.

In other embodiments, as shown in FIG. 2D, the concession slot includes an opening facing a proximal direction of the accommodating tube 400. In this case, the concession slot 430 penetrates the proximal edge of the accommodating tube 400, thereby forming the opening, while an end of the concession slot 430 located at the middle portion of the accommodating tube 400 is closed. The tube body of the accommodating tube 400 located between the concession slot 430 and the slot 410 forms a cantilever beam structure. A distal end of the tube body of the accommodating tube 400 located between the concession slot 430 and the slot 410 may be regarded as a support point of the cantilever beam structure. Through such a configuration, the tube body of the accommodating tube 400 (the portion of the tube body between the concession slot 430 and the slot 410) may be deformed more easily, more effectively avoiding excessive resistance to movement of the locking portion 110 within at least certain sections of the slot 410 (e.g., the first slot 413).

In some embodiments, as shown in FIGS. 2B-2D, the slot 410 further includes the first slot 413. The first slot 413 is located between the sliding slot 411 and the locking slot 412. One end (e.g., a distal end) of the first slot 413 is connected to the sliding slot 411, and the other end (e.g., a proximal end) of the first slot 413 is connected to the locking slot 412. The first slot 413 is a part of the slot 410. The sliding slot 411, the first slot 413, and the locking slot 412 may be arranged sequentially along the axial direction of the slot 410. The locking portion 110 may achieve a positional change between the sliding slot 411 and the locking slot 412 through the first slot 413. For example, when the locking portion 110 is located within the sliding slot 411, the locking portion 110 moves toward the proximal direction, and enters the locking slot 412 after passing through the first slot 413. As another example, when the locking portion is located within the locking slot 412, the locking portion 110 moves toward the distal direction, and enters the sliding slot 411 after passing through the first slot 413.

In some embodiments, the first slot 413 includes a third limiting portion 4131 that limits the movement of the locking portion 110 from the locking slot 412 toward the distal direction. In some embodiments, a slot wall at an end of the first slot 413 connected to the locking slot 412 constitutes the third limiting portion 4131. A structure of the third limiting portion 4131 is further described below.

In some embodiments, the slot wall of the first slot 413 directly constitutes the third limiting portion 4131 by configuring a slot width of the first slot 413. In some embodiments, a size of the first slot 413 in a first direction is less than a size of the locking portion 110 in the first direction. It should be noted that the first direction may be understood as the circumferential direction of the accommodating tube 400. The size of the first slot 413 in the first direction may be understood as a maximum slot width of the first slot 413. The size of the locking portion 110 in the first direction may be understood as a width of the locking portion 110. By setting the size of the first slot 413 in the first direction to be less than the size of the locking portion 110 in the first direction, the slot wall of the first slot 413 may limit and hinder the movement of the locking portion 110, thereby limiting the movement of the locking portion 110 from the locking slot 412 toward the distal direction. When the locking portion 110 is fitted within the first slot 413 (at this time, the locking portion 110 has entered the first slot 413), the locking portion 110 squeezes the slot wall of the first slot 413. Due to the presence of the concession slot 430, the tube body of the accommodating tube 400 (the portion of the tube body between the concession slot 430 and the slot 410) may be deformed, thereby avoiding excessive resistance to the movement of the locking portion 110 within the first slot 413.

In some embodiments, a slot width at an end of the first slot 413 connected to the locking slot 412 in the first direction is set to be less than a slot width at an end of the locking slot 412 connected to the first slot 413 in the first direction, so that the slot wall at the end of the first slot 413 connected to the locking slot 412 constitutes the third limiting portion 4131. Through such a configuration, the slot wall at the end of the first slot 413 connected to the locking slot 412 may limit and hinder the locking portion 110 from leaving the locking slot 412.

In some other embodiments, the third limiting portion 4131 is constituted by specially designing a structure of the slot wall at the end of the first slot 413 connected to the locking slot 412. For example, the slot wall at the end of the first slot 413 connected to the locking slot 412 includes a projected structure that constitutes the third limiting portion 4131, and the projected structure also limits and hinders the locking portion 110 from leaving the locking slot 412. As another example, the slot wall at the end of the first slot 413 connected to the locking slot 412 includes an elastic flap that constitutes the third limiting portion 4131, and the elastic flap also limits and hinders the locking portion 110 from leaving the locking slot 412.

In some embodiments, setting the size of the first slot 413 in the first direction to be less than the size of the locking portion 110 in the first direction makes the distal end of the first slot 413 form a stop against the locking portion 110 when the locking portion 110 is located at a proximal end of the sliding slot 411. During the operation of the clip arm 100, when the locking portion 110 moves to the proximal end of the sliding slot 411, the distal end of the first slot 413 forms the stop against the locking portion 110. The locking portion 110 then receives a feedback resistance applied by the distal end of the first slot 413. The feedback resistance may be fed back to the control portion 300, thereby reminding an operator that the locking portion 110 is about to enter the first slot 413. After obtaining the feedback resistance, the operator may reconfirm a clamping situation of the clip portion 120 on a target object. If the clip portion 120 satisfies a clamping requirement, the operator increases a driving force controlling the clip arm 100 to move toward the proximal direction, and causes the locking portion 110 to enter the first slot 413. If the clip portion 120 does not satisfy the clamping requirement, the operator controls the clip arm 100 to move toward the distal direction, and makes the clip portion 120 to re-open.

As the slot wall at the end of the first slot 413 connected to the locking slot 412 constitutes the third limiting portion 4131, and the slot width of the first slot 413 is less than the width of the locking portion 110, compared to the locking portion 110 being located outside the first slot 413, when the locking portion 110 is located within the first slot 413, the locking portion 110 squeezes the slot wall of the first slot 413, thereby causing the tube body of the accommodating tube 400 (the portion of the tube body between the concession slot 430 and the slot 410) to deform toward the concession slot 430. At this time, a slot width of at least the portion of the concession slot 430 in the circumferential direction of the accommodating tube 400 becomes smaller. Simultaneously, the slot width of the first slot 413 in the circumferential direction of the accommodating tube 400 (the first direction) increases, thereby avoiding excessive resistance to the movement of the locking portion 110 within the first slot 413. When the locking portion 110 is located outside the first slot 413, the tube body of the accommodating tube 400 (the portion of the tube body between the concession slot 430 and the slot 410) may not deform or may only deform slightly toward the concession slot 430.

It should be noted that when the locking portion 110 is located within the sliding slot 411 and the locking slot 411, the tube body of the accommodating tube 400 (the portion of the tube body between the concession slot 430 and the slot 410) may also deform toward the concession slot 430, but an amount of deformation of the tube body of the accommodating tube 400 at this time is less than an amount of deformation of the tube body of the accommodating tube 400 when the locking portion 110 is located within the first slot 413.

In some embodiments, a proximal edge of the concession slot 430 is closer to the proximal edge of the accommodating tube 400 than the third limiting portion 4131, and a distal edge of the concession slot 430 is closer to a distal edge of the accommodating tube 400 than the third limiting portion 4131. That is, a position where the third limiting portion is located falls within a range of a first length region covered by the concession slot 430 along the axial direction of the accommodating tube 400. When the locking portion 110 moves to a position corresponding to the third limiting portion 4131, the locking portion 110 squeezes the slot wall of the first slot 413, and at this time, a squeezing force of the locking portion 110 on the slot wall of the first slot 413 is the greatest. By setting the relative positions of the third limiting portion 4131 and the concession slot 430 as described above, the tube body of the accommodating tube 400 (the portion of the tube body between the concession slot 430 and the slot 410) is prone to deformation when the locking portion 110 moves to the position corresponding to the third limiting portion 4131, thereby avoiding the excessive resistance of the locking portion 110 at the third limiting portion 4131, and preventing the locking portion 110 from having difficulty overcoming the limitation of the third limiting portion 4131 to release the locked state.

In some embodiments, the distal edge of the concession slot 430 is closer to the distal edge of the accommodating tube 400 than the proximal edge of the first slot 413, and the proximal edge of the concession slot 430 is closer to the proximal edge of the accommodating tube 400 than the distal edge of the first slot 413. At this time, a second length region covered by the first slot 413 along the axial direction of the accommodating tube 400 at least partially overlaps the first length region covered by the concession slot 430 along the axial direction of the accommodating tube 400. By setting the relative position of the first slot 413 and the concession slot 430 as described above, the tube body of the accommodating tube 400 (the portion of the tube body between the concession slot 430 and the slot 410) may deform when the locking portion 110 moves into the first slot 413 (e.g., when the locking portion 110 is at a position corresponding to the third limiting portion 4131), thereby avoiding the excessive resistance of the locking portion 110 within the first slot 413.

In some embodiments, the proximal edge of the concession slot 430 is closer to the proximal edge of the accommodating tube 400 than the proximal edge of the first slot 413, and the distal edge of the concession slot 430 is closer to the distal edge of the accommodating tube 400 than the distal edge of the first slot 413. That is, the second length region covered by the first slot 413 along the axial direction of the accommodating tube 400 is within the range of a first length region covered by the concession slot 430 along the axial direction of the accommodating tube 400. As the slot wall of the first slot 413 constitutes the third limiting portion 4131, by setting the relative position of the first slot 413 and the concession slot 430 as described above, the tube body of the accommodating tube 400 (the portion of the tube body between the concession slot 430 and the slot 410) easily deforms when the locking portion 110 moves into the first slot 413, thereby avoiding the excessive resistance of the locking portion 110 within the first slot 413.

By controlling the clip arm 100 (e.g., through the operation portion 300), the locking portion 110 may be controlled to slide within the sliding slot 411, so as to switch the clip arm 100 between the open state and the closed state. When the locking portion 110 is controlled to move from the distal end toward the proximal direction within the sliding slot 411, the clip arm 100 is switched from the open state to the closed state. Conversely, when the locking portion 110 is controlled to move from the proximal direction toward the distal direction within the sliding slot 411, the clip arm 100 is switched from the closed state to the open state. When the locking portion 110 is controlled to move toward the proximal direction and enters the first slot 413 from the sliding slot 411, the clip arm 100 maintains the closed state while the locking portion 110 is located within the first slot 413. When the locking portion 110 is controlled to continue moving toward the proximal end and enters the locking slot 412 from the first slot 413, the clip arm 100 is in the locked state while the locking portion 110 is located within the locking slot 412. Therefore, by controlling the clip arm 100, a position change of the locking portion 110 within the slot 410 may be controlled, thereby switching the clip arm 100 among the open state, the closed state, and the locked state. In addition, due to the arrangement of the third limiting portion 4131, the movement of the locking portion 110 from the locking slot 412 toward the distal direction is also limited.

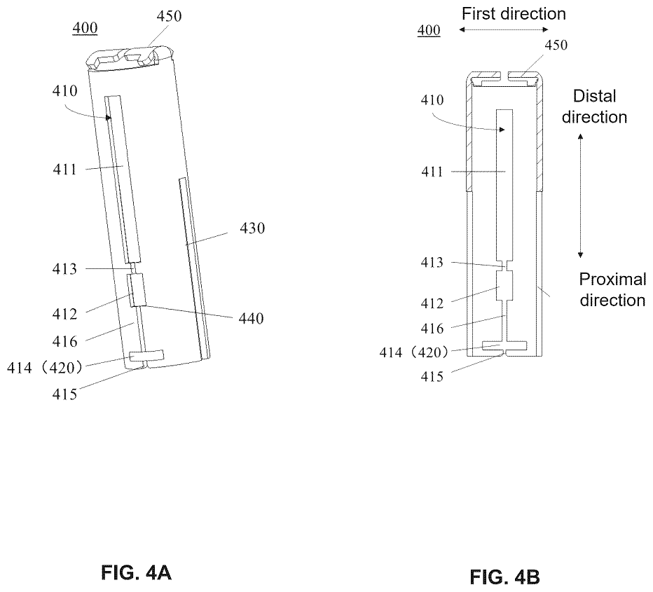

It should be noted that, in different embodiments of the present disclosure, a function of the first slot 413 may not be completely identical, and the first slot 413 may also have a plurality of functions. For example, in the embodiments shown in FIGS. 3A-4B below, by setting the locking portion 110 to be located in the first slot 413, the accommodating tube 400 and the sheath 210 are released. In this embodiment, the slot wall of the first slot 413 still functions to limit the locking portion 110 from moving toward the distal direction and leaving the locking slot 412.

FIG. 3A is a structural sectional diagram illustrating an exemplary clip instrument 10 according to some embodiments of the present disclosure. FIG. 3B is a partial enlarged diagram illustrating region B of the clip instrument 10 shown in FIG. 3A. FIG. 4A is a structural diagram illustrating an exemplary accommodating tube 400 according to some embodiments of the present disclosure. FIG. 4B is a sectional diagram illustrating an exemplary accommodating tube 400 taken along an axial direction according to some embodiments of the present disclosure;

As shown in FIGS. 3A-4B, in some embodiments, the clip instrument 10 further includes the sheath 210, and the accommodating tube 400 is releasably connected to the sheath 210. The “releasably connected” in the embodiments of the present disclosure refers to two components maintaining a connected state when a preset condition is satisfied (e.g., when the two components form a limiting fit), and separating from each other when the preset condition is not satisfied (e.g., when the limiting fit between the two components is disengaged). In some embodiments, after the accommodating tube 400 and the sheath 210 are released, the accommodating tube 400 and the clip arm 100 remain at a target object. For example, the accommodating tube 400 and the clip arm 100 are retained at a wound position to achieve hemostasis, while other components such as the sheath 210 and the mandrel 220 are withdrawn from a human body.

In some embodiments, the slot 410 further includes the first slot 413. The first slot 413 is located between the sliding slot 411 and the locking slot 412. When the locking portion 110 of the clip arm 100 is located in the first slot 413, the accommodating tube 400 and the sheath 210 are released. By locating the first slot 413 between the sliding slot 411 and the locking slot 412, the locking portion 110 passes through the first slot 413 before entering the locking slot 412. Therefore, during a process where the clip arm 100 moves from a distal direction toward a proximal direction, the accommodating tube 400 and the sheath 210 are released first, and then the clip arm 100 is locked with the accommodating tube 400. This sequence ensures that the clip arm 100 no longer continues to move after being locked, thereby guaranteeing the reliability of the locking. In summary, by providing the slot 410, a release process and a locking process of the clip arm 100 may be achieved through the simple slot 410. The operation is simple, which both maintains the locking reliability and makes the release of the accommodating tube 400 more convenient.

In some embodiments, the sheath 210 includes a first connection structure 211, and the accommodating tube 400 includes a second connection structure 420. The first connection structure 211 and the second connection structure 420 are releasably connected. In some embodiments, one of the first connection structure 211 and the second connection structure 420 includes a connection projection, and the other includes a connection slot. In some other embodiments, the first connection structure 211 and the second connection structure 420 also include other connection forms.

In some embodiments, when the locking portion 110 of the clip arm 100 is located in the first slot 413, the second connection structure 420 is actuated to undergo displacement, causing the first connection structure 211 and the second connection structure 420 to disengage. By moving the locking portion 110 to the first slot 413, the first connection structure 211 and the second connection structure 420 may be quickly disengaged, making a control process of the clip instrument 10 more convenient and efficient.

In some embodiments, the first connection structure 211 includes a connection projection 2111. In some embodiments, the connection projection 2111 is provided on an end face at a distal end of the sheath 210. Alternatively, the connection projection 2111 is provided on other components within the sheath 210. For example, the connection projection 2111 is provided on an elastic member 212 within the sheath 210.

In some embodiments, the slot 410 further includes a connection slot 414. The connection slot 414 is located at a proximal direction of the locking slot 412. The connection slot 414 constitutes the second connection structure 420. The connection projection 2111 and the connection slot 414 are releasably connected. For example, when the locking portion 110 is engaged in the first slot 413, the locking portion 110 actuates the connection slot 414 to undergo displacement (e.g., the connection slot 414 moves as a whole or a side wall of the connection slot 414 deforms to cause the displacement), causing the connection slot 414 and the connection projection 2111 to disengage.

In some embodiments, a size of the first slot 413 in a first direction is smaller than a size of the locking portion 110 in the first direction. The first direction refers to a direction indicated by an arrow in FIG. 4B. Alternatively, the first direction may be understood as a circumferential direction of the accommodating tube 400. When the locking portion 110 is engaged in the first slot 413, the first slot 413 expands in the circumferential direction, causing the accommodating tube 400 to undergo an elastic deformation in the circumferential direction. This further causes the connection slot 414 to undergo displacement, making the connection slot 414 and the connection projection 2111 to disengage. At this time, by operating the sheath 210 to move toward the proximal direction or operating the connection projection 2111 to move toward the proximal direction, the connection projection 2111 retracts from the connection slot 414, and the accommodating tube 400 and the sheath 210 are released.

In some embodiments, a size of the sliding slot 411 in the first direction is greater than or equal to the size of the locking portion 110 in the first direction. In this way, a frictional resistance of the sliding slot 411 against the locking portion 110 is relatively small, making opening and closing processes of the clip arm 100 smoother. In some embodiments, a size of the locking slot 412 in the first direction is greater than or equal to the size of the locking portion 110 in the first direction. In this way, after the locking portion 110 enters the locking slot 412 from the first slot 413, the accommodating tube 400 recovers to its original shape. At this time, the proximal end of the first slot 413 may limit the locking portion 110 from moving toward the distal direction, and the proximal end of the locking slot 412 may limit the locking portion 110 from moving toward the proximal direction.

During an operation process of the clip arm 100, when the locking portion 110 moves to the proximal end of the sliding slot 411, the distal end of the first slot 413 forms a stop against the locking portion 110. Then, the locking portion 110 receives a feedback resistance applied by the distal end of the first slot 413. The feedback resistance may be fed back to the control portion 300 and used to remind an operator that the accommodating tube 400 is about to be released from the sheath 210, or that the clip arm 100 is about to be locked. After obtaining the feedback resistance, the operator may reconfirm a clamping condition of the clip portion 120 on the target object. If the clip portion 120 satisfies a clamping requirement, the operator increases a driving force for controlling the clip arm 100 to move toward the proximal direction, making the locking portion 110 to enter the first slot 413 and perform a release operation between the accommodating tube 400 and the sheath 210. If the clip portion 120 does not satisfy the clamping requirement, the operator controls the clip arm 100 to move toward the distal direction, making the clip portion 120 to re-open.

In some embodiments, the slot 410 further includes a second slot 415. The second slot 415 extends from the proximal end of the connection slot 414 along the axial direction of the accommodating tube 400 to the proximal edge of the accommodating tube 400. The second slot 415 is configured to allow the connection projection 2111 to enter and retract from the connection slot 414.

In some embodiments, when the locking portion 110 is engaged in the first slot 413, the accommodating tube 400 deforms such that a size of the second slot 415 in the first direction becomes greater than or equal to a size of the connection projection 2111 in the first direction. The connection projection 2111 and the connection slot 414 disengage, and at this time, the connection projection 2111 freely enters and retracts from the connection slot 414. By controlling the connection projection 2111 to move toward the proximal direction, the connection projection 2111 retracts from the connection slot 414, and the accommodating tube 400 and the sheath 210 are released. By actuating the first slot 413 through the locking portion 110, a side wall of the accommodating tube 400 undergoes an elastic deformation, thereby causing an increase in the size of the first slot 413 in the first direction and disengaging an engagement relationship between the connection projection 2111 and the connection slot 414. This improves the convenience of releasing the accommodating tube 400 and the sheath 210.

In some embodiments, when the locking portion 110 is not engaged in the first slot 413, a size of the second slot 415 in the first direction is smaller than the size of the connection projection 2111 in the first direction. A proximal end of the connection slot 414 limits the movement of the connection projection 2111 toward the proximal direction, and the accommodating tube 400 is connected to the sheath 210.

In some embodiments, the slot 410 further includes a third slot 416. The third slot 416 extends from the locking slot 412 to the connection slot 414. By providing the third slot 416, when the locking portion 110 is engaged in the first slot 413, the deformation of the first slot 413 causes the deformation of the second slot 415, thereby causing a change in a size of the second slot 415 in a certain direction. In some embodiments, a proximal end of the locking slot 412 needs to form a limit against the locking portion 110. Therefore, a size of the third slot 416 in the first direction is smaller than the size of the locking portion 110 in the first direction, to prevent the locking portion 110 from entering the third slot 416. For example, the third slot 416 is an axial slit formed on a side wall of the accommodating tube 400 by means such as cutting.

In some embodiments, the accommodating tube 400 further includes at least one concession slot 430. The at least one concession slot 430 extends from a middle portion of the accommodating tube 400 to the proximal edge of the accommodating tube 400. The at least one concession slot 430 and the slot 410 are spaced apart along the circumferential direction of the accommodating tube 400. By providing the at least one concession slot 430, the side wall of the accommodating tube 400 deforms more easily, thereby avoiding an excessive resistance to the movement of the locking portion 110 within the first slot 413. It should be noted that the at least one concession slot may have a closed-end structure or an open-end structure, as long as the at least one concession slot facilitates the deformation of the side wall of the accommodating tube.

In some embodiments, the second connection structure 420 includes a connection recess disposed within the first slot 413. The first connection structure 211 includes a connection arm extending from the distal end of the sheath 210. A distal end of the connection arm includes a hook. The hook engages with the connection recess. When the locking portion 110 of the clip arm 100 enters the first slot 413, the locking portion 110 squeezes the hook to cause displacement of the hook, thereby disengaging the hook from the connection recess and releasing the accommodating tube 400 and the sheath 210.

In some embodiments, the sheath 210 includes the elastic member 212 disposed within a passage of the sheath 210. A proximal end of the elastic member 212 is connected to an inner wall of the sheath 210. A distal end of the elastic member 212 is axially movably disposed at the distal end of the sheath 210. The first connection structure 211 is disposed at the distal end of the elastic member 212. In some embodiments, the elastic member 212 includes a spring 2121 and a spring tip 2122. The spring tip 2122 is fixed to a distal end of the spring 2121. The first connection structure 211 is disposed on the spring tip 2122. The spring tip 2122 engages with the second connection structure 420 through the first connection structure 211. In other embodiments, the elastic member 212 is in other forms, for example, a rubber ring, etc.

In some embodiments, when the second connection structure 420 engages with the first connection structure 211, the elastic member 212 is in a stretched state. For example, the elastic member 212 is in the stretched state. The elastic member 212 applies a pulling force toward the proximal direction to the first connection structure 211. As the first connection structure 211 and the second connection structure 420 form a limiting engagement, the elastic member 212 remains in the stretched state before the accommodating tube 400 and the sheath 210 are released.

In some embodiments, when the engagement between the second connection structure 420 and the first connection structure 211 is released, the elastic member 212 drives the second connection structure 420 and the first connection structure 211 to separate, thereby releasing the accommodating tube 400 and the sheath 210. In some embodiments, when the locking slot 412 engages with the first slot 413, the accommodating tube 400 deforms. A size of the second slot 415 in the first direction is greater than or equal to the size of the connection projection 2111 in the first direction. Therefore, the connection projection 2111 is no longer constrained by the second slot 415. Under an elastic restoring force of the elastic member 212, the elastic member 212 pulls the connection projection 2111 to move toward the proximal direction, causing the connection projection 2111 to disengage from the connection slot 414. Accordingly, the second connection structure 420 separates from the first connection structure 211, and the accommodating tube 400 and the sheath 210 are released. By providing the elastic member 212 to drive the first connection structure 211 to rapidly separate from the second connection structure 420, the efficiency and the reliability of releasing the accommodating tube 400 and the sheath 210 are improved.

In some embodiments, a driving force value F1 for movement of the locking portion 110 within the first slot 413 is in a range of 30 N≤F1<50 N. For example, the driving force value F1 includes, but is not limited to, 30 N, 35 N, 40 N, 45 N, etc. When the driving force value F1 of the locking portion 110 is within the above range, a phenomena of unstable movement caused by an excessively great or excessively small driving force, such as jamming or excessively fast movement, may be avoided. The locking portion 110 moves within the first slot 413 at a more reasonable speed to ensure that the elastic member 212 pulls the connection projection 2111 out of the connection slot 414 before the locking portion 110 enters the locking slot 412 for locking. The stability and the accuracy of the operation process are thereby ensured.

In some embodiments, a driving force value F2 for the movement of the locking portion 110 within the sliding slot 411 is in a range of 0 N<F2<30 N. For example, the driving force value F2 includes, but is not limited to, 5 N, 10 N, 15 N, 20 N, 25 N, etc. The driving force value F2 is less than the driving force value F1. Therefore, when the locking portion 110 engages with the sliding slot 411, only a relatively small driving force is required to move the locking portion 110 toward the distal direction or toward the proximal direction. When the locking portion 110 needs to engage with the first slot 413, the driving force is increased to be in a range of 30 N≤F1<50 N to cause the locking portion 110 to move from the sliding slot 411 into the first slot 413. The operation is simple and convenient.

In some embodiments, the accommodating tube 400 is rotatably disposed at the distal direction of the sheath 210 about an axis of the sheath 210, thereby facilitating an alignment of a closing direction of the clip portion 120 with a closing direction of the target object. In some embodiments, when the first connection structure 211 engages with the second connection structure 420, the first connection structure 211 and the second connection structure 420 are rotatable about the axis of the sheath 210 (or the axis of the accommodating tube 400). In some embodiments, the connection slot 414 extends along the circumferential direction of the accommodating tube 400, allowing the connection projection 2111 to move relative to the circumferential direction of the accommodating tube 400, thereby enabling the accommodating tube 400 to rotate about the axis of the sheath 210. In some embodiments, a length of the connection slot 414 is configured such that a rotation angle α of the accommodating tube 400 about the axis of the sheath 210 is within a range of 60°-150°. For example, the length of the connection slot 414 is configured such that the accommodating tube 400 is rotatable about the axis of the sheath 210 by 90°.

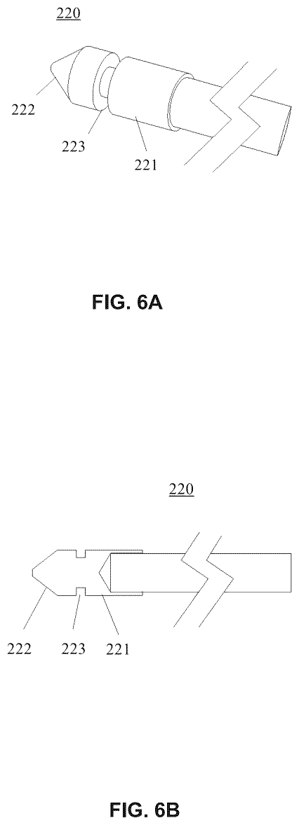

FIG. 5A is an exemplary structural diagram illustrating the clip arm 100 according to some embodiments of the present disclosure, wherein the connection pin 130 and the clip portion 120 are shown in a disassembled state. FIG. 5B is an exemplary front view illustrating the clip arm 100 according to some embodiments of the present disclosure. FIG. 6A is an exemplary structural diagram illustrating the mandrel 220 according to some embodiments of the present disclosure. FIG. 6B is an exemplary structural diagram illustrating the mandrel 220 according to some embodiments of the present disclosure.

As shown in FIGS. 5A-6B, in some embodiments, the clip arm 100 includes at least two clip portions 120. Each of the at least two clip portions 120 includes a distal joint portion 121, a bending portion 122, and a proximal joint portion 123. The distal joint portion 121 refers to a portion for clamping tissue. The distal joint portion 121 is always located outside the accommodating tube 400. The bending portion 122 is elastic. After the bending portion 122 enters the accommodating tube 400, the bending portion 122 elastically deforms due to a spatial constraint of the accommodating tube 400, causing the distal joint portion 121 to close. After the bending portion 122 extends out of the accommodating tube 400, the bending portion 122 bends under its own elastic restoring force, causing the distal joint portion 121 to open. The proximal joint portion 123 is always located inside the accommodating tube 400 and is configured for a releasable connection with the mandrel 220.

In some embodiments, the proximal joint portions 123 of the at least two clip portions 120 are integrally formed, improving the stability and the symmetry of the at least two clip portions 120 and thereby improving the clamping stability.

In some embodiments, the proximal joint portion 123 includes a first connection portion 125. The first connection portion 125 includes a pin hole 1251. The clip arm 100 includes a connection pin 130. The connection pin 130 is connected to the pin hole 1251. The connection pin 130 improves the stability and the strength of the proximal joint portion 123. In some embodiments, two end portions of the connection pin 130 constitute the locking portion 110. The accommodating tube 400 includes two slots 410. The locking portions 110 at the two ends of the connection pin 130 respectively engage with the two slots 410.

In some embodiments, the proximal joint portion 123 includes a second connection portion 126. The second connection portion 126 includes a connection hole 1261. The clip instrument 10 includes the mandrel 220. A distal end of the mandrel 220 is provided with a connection tip 221. The connection tip 221 is releasably connected to the connection hole 1261. In some embodiments, the connection tip 221 includes a guiding inclined surface 222 and a limiting concession slot 223 located at a proximal end of the guiding inclined surface 222. The guiding inclined surface 222 is configured to guide the connection tip 221 into the connection hole 1261. The connection hole 1261 engages with the limiting concession slot 223, thereby enabling the releasable connection between the connection tip 221 and the clip arm 100.

In some embodiments, the proximal joint portion 123 further includes an installation slot 127. The installation slot 127 extends from the connection hole 1261 to the first connection portion 125. By providing the installation slot 127, when the connection tip 221 is assembled with or disassembled from the proximal joint portion 123, the proximal joint portion 123 elastically deforms and expands outward from the installation slot 127, allowing the connection tip 221 to enter or retract from the connection hole 1261. After the assembly or disassembly is completed, the proximal joint portion 123 returns to its original shape.

Referring to FIG. 4A, in some embodiments, the slot 410 includes a first limiting portion 440. The first limiting portion 440 is disposed in a proximal direction of the locking slot 412. In some embodiments, the size of the locking slot 412 in the first direction is greater than the size of the third slot 416 in the first direction. A step is formed between the locking slot 412 and the third slot 416. The step is configured as the first limiting portion 440.

In some embodiments, after the locking portions 110 at the two ends of the connection pin 130 abut against and are limited by the first limiting portion 440, the mandrel 220 moves from the distal direction toward the proximal direction. The mandrel 220 drives the connection tip 221 to disengage from the connection hole 1261, thereby releasing the clip arm 100 from the mandrel 220.

In some embodiments, a driving force value F3 for disengaging the connection tip 221 from the connection hole 1261 is in a range of 60 N<F3<80 N. For example, the driving force value F3 includes, but is not limited to, 65 N, 70 N, 75 N, etc. The driving force value F3 is greater than the driving force values F1 and F2 of the locking portion 110 within the slot 410. Therefore, after the accommodating tube 400 and the sheath 210 are released and the clip arm 100 is locked with the accommodating tube 400, the connection tip 221 is released from the clip arm 100. In this way, an operational smoothness is ensured, and an operational efficiency is improved.

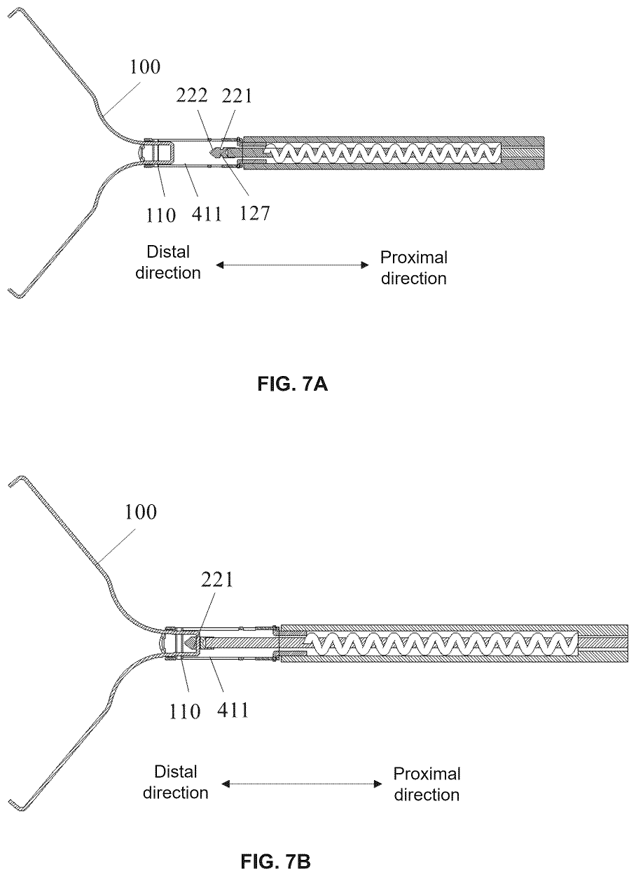

FIG. 7A is an exemplary structural diagram illustrating the connection tip 221 of the clip instrument 10 and the clip arm 100 in a disconnected state according to some embodiments of the present disclosure. FIG. 7B is an exemplary structural diagram illustrating the connection tip 221 of the clip instrument 10 and the clip arm 100 in a connected state according to some embodiments of the present disclosure;

As shown in FIG. 5A to FIG. 7A, in some embodiments, the connection tip 221 and the clip arm 100 are in the disconnected state. The clip arm 100 is in an open state. The locking portion 110 of the clip arm 100 is located at a distal end of the sliding slot 411. The distal end of the sliding slot 411 limits the locking portion 110, preventing further movement of the clip arm 100 toward the distal direction. The control portion 300 controls the connection tip 221 to move from the proximal direction toward the distal direction, causing the guiding inclined surface 222 of the connection tip 221 to extend into the connection hole 1261 of the clip arm 100. As the guiding inclined surface 222 moves toward the distal direction, the connection hole 1261 is gradually expanded, allowing the connection tip 221 to gradually enter the connection hole 1261.

As shown in FIGS. 5A-6B and FIG. 7B, in some embodiments, the connection tip 221 and the clip arm 100 are in the connected state. The connection tip 221 continues to move from the distal direction toward the proximal direction. An edge of the connection hole 1261 of the clip arm 100 embeds into the installation slot 127 of the connection tip 221. At this point, the connection tip 221 and the connection hole 1261 form a limiting engagement, and the mandrel 220 is releasably connected to the clip arm 100.

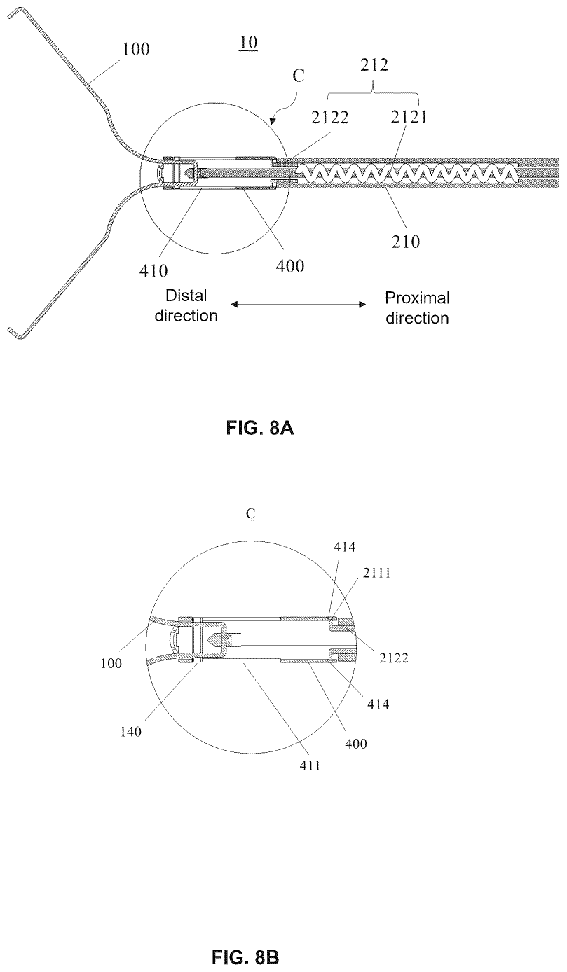

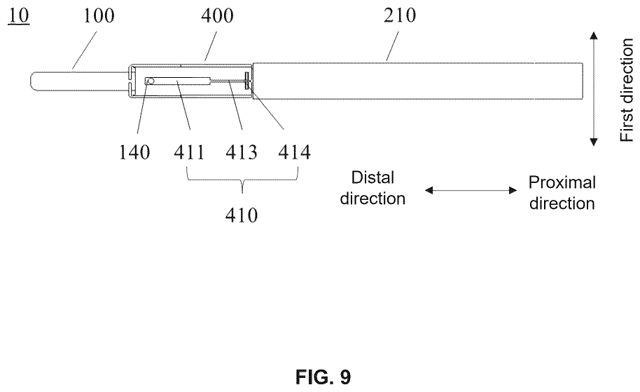

FIG. 8A is a diagram illustrating an exemplary sectional structure of a clip instrument 10 according to some embodiments of the present disclosure. FIG. 8B is a partial enlarged diagram illustrating a region C of the clip instrument 10 shown in FIG. 8A. FIG. 9 is an exemplary structural diagram illustrating the clip instrument 10 according to some embodiments of the present disclosure.

As shown in FIGS. 8A-9, Embodiment 2 of the present disclosure provides the clip instrument 10. The clip instrument 10 includes the clip arm 100, the accommodating tube 400, and the sheath 210.

In some embodiments, a proximal end of the clip arm 100 is provided with a sliding portion 140. The proximal end of the clip arm 100 is movably disposed in the accommodating tube 400. The accommodating tube 400 includes at least one slot 410 extending along an axial direction. The slot 410 includes a sliding slot 411, the first slot 413, and the connection slot 414. The sliding slot 411 is located at a distal direction of the first slot 413. The connection slot 414 is located at a proximal direction of the first slot 413. A difference between the clip instrument 10 of Embodiment Two and the clip instrument 10 of Embodiment One is that, in Embodiment Two, a slot is not configured with a locking slot. Locking of the clip arm 100 may be performed by the accommodating tube 400 itself. Alternatively, the clip arm 100 may be locked by setting an independent locking member.

In some embodiments, the sliding portion 140 slidably engages with the slot 410, enabling the proximal end of the clip arm 100 to move relative to the accommodating tube 400. When the sliding portion 140 engages with the sliding slot 411, the clip instrument 10 switches between an open state and a closed state. For example, when the sliding portion 140 moves toward the distal direction within the sliding slot 411, the clip arm 100 extends out of the accommodating tube 400 and presents the open state. When the sliding portion 140 moves toward the proximal direction within the sliding slot 411, the clip arm 100 retracts into the accommodating tube 400 and presents the closed state.

In some embodiments, the sheath 210 includes a connection projection 2111. The connection projection 2111 is releasably connected to the connection slot 414. When the sliding portion 140 is located in the first slot 413, the connection projection 2111 disengages from the connection slot 414, and the accommodating tube 400 and the sheath 210 are released.

According to the solution in the above embodiments, a side wall of the accommodating tube 400 includes the slot 410. The sliding slot 411, the first slot 413, and the connection slot 414 are all concentrated in the slot 410, thereby simplifying an overall structure. Furthermore, by making the sliding portion 140 engage with the first slot 413, the connection slot 414 and the connection projection 2111 may disengage, thereby making a process of releasing the accommodating tube 400 and the sheath 210 more convenient.

More exemplary embodiments of the clip instrument 10 in Embodiment Two will be described below in conjunction with FIGS. 8A-9. It should be understood that certain features, structures, or characteristics in Embodiment Two may refer to corresponding features, structures, or characteristics in Embodiment One. The present disclosure does not repeat descriptions of some identical features. It should be noted that descriptions of some features in Embodiment Two may be referred to in FIGS. 1A-7B and related descriptions thereof.

In some embodiments, a size of the first slot 413 in a first direction is smaller than a size of the sliding portion 140 in the first direction. The first direction refers to a direction indicated by an arrow in FIG. 9. Alternatively, the first direction may be understood as a circumferential direction of the accommodating tube 400. When the sliding portion 140 engages with the first slot 413, the first slot 413 expands along the circumferential direction, causing the accommodating tube 400 to undergo an elastic deformation in the circumferential direction. This further causes the connection slot 414 to displace, to disengage the connection slot 414 and the connection projection 2111. At this time, by operating the sheath 210 to move toward the proximal direction or operating the connection projection 2111 to move toward the proximal direction, so that the connection projection 2111 retracts from the connection slot 414, and the accommodating tube 400 and the sheath 210 are released.

In some embodiments, a size of the sliding slot 411 in the first direction is greater than or equal to a size of the sliding portion 140 in the first direction. In this way, a frictional resistance of the sliding slot 411 against the sliding portion 140 is relatively small, making opening and closing processes of the clip arm 100 smoother.

In some embodiments, the slot 410 further includes the second slot 415. The second slot 415 extends from a proximal end of the connection slot 414 along the axial direction of the accommodating tube 400 to a proximal edge of the accommodating tube 400. The second slot 415 is configured to allow the connection projection 2111 to enter and retract from the connection slot 414.

In some embodiments, when the sliding portion 140 engages with the first slot 413, the accommodating tube 400 deforms such that a size of the second slot 415 in the first direction becomes greater than or equal to a size of the connection projection 2111 in the first direction. The connection projection 2111 disengages from the connection slot 414. At this time, the connection projection 2111 freely enters and retracts from the connection slot 414. When the connection projection 2111 is controlled to move toward the proximal direction, the connection projection 2111 retracts from the connection slot 414, and the accommodating tube 400 and the sheath 210 are released. By actuating the first slot 413 with the sliding portion 140, a side wall of the accommodating tube 400 undergoes the elastic deformation, thereby causing the size of the first slot 413 in the first direction to increase and disengaging the engagement relationship between the connection projection 2111 and the connection slot 414, thereby improving the convenience of releasing the accommodating tube 400 and the sheath 210.

In some embodiments, when the sliding portion 140 does not engage with the first slot 413, the size of the second slot 415 in the first direction is smaller than the size of the connection projection 2111 in the first direction. A proximal end of the connection slot 414 limits a movement of the connection projection 2111 toward the proximal direction, and the accommodating tube 400 is connected to the sheath 210.

In some embodiments, the accommodating tube 400 further includes at least one concession slot 430. The concession slot 430 extends from a middle portion of the accommodating tube 400 to the proximal edge of the accommodating tube 400. The concession slot 430 and the slot 410 are spaced apart along the circumferential direction of the accommodating tube 400. A portion of the accommodating tube 400 located between the concession slot 430 and the slot 410 forms a cantilever. When the sliding portion 140 engages with the first slot 413, the cantilever deforms and displaces, causing the size of the second slot 415 in the first direction to change, and the connection projection 2111 and the connection slot 414 disengage. By providing the concession slot 430, the side wall of the accommodating tube 400 deforms more easily, avoiding an excessive resistance to the movement of the sliding portion 140 within the first slot 413.

In some embodiments, the accommodating tube 400 locks the clip arm 100. For example, when the bending portion 122 of the clip arm 100 is contained within the accommodating tube 400, the accommodating tube 400 limits the opening of the clip arm 100 from the circumferential direction, while a frictional force between the accommodating tube 400 and the clip arm 100 along the axial direction prevents the clip arm 100 from detaching from the accommodating tube 400, thereby achieving the locking function. In some other embodiments, the clip instrument 10 further includes the locking member. The proximal end of the clip arm 100 is provided with a to-be-locked portion. The to-be-locked portion and the locking member form a limiting engagement to lock the clip arm 100. For example, the locking member is disposed at a distal end of the sheath 210 or disposed inside the accommodating tube 400. The locking member is a block structure with a locking cavity. The locking member is formed with a limiting recess. The to-be-locked portion of the clip arm 100 is formed with a limiting projection. After the proximal end of the clip arm 100 enters the locking cavity, the limiting recess and the limiting projection engage, thereby achieving locking. As another example, the clip arm 100 includes at least two clip portions 120. A side edge of at least one clip portion 120 forms the locking projection. A side edge of at least another clip portion 120 forms the locking recess. The locking projection and the locking recess engage, thereby locking the at least two clip portions 120.

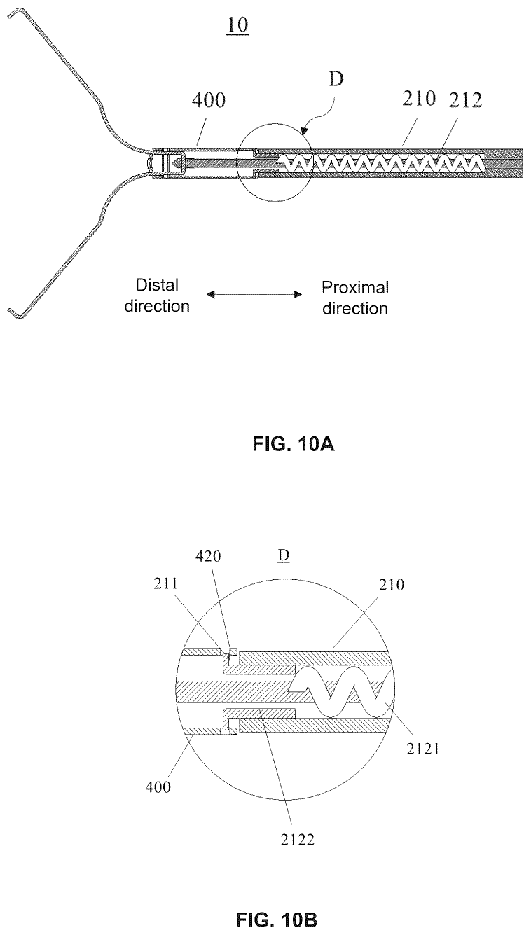

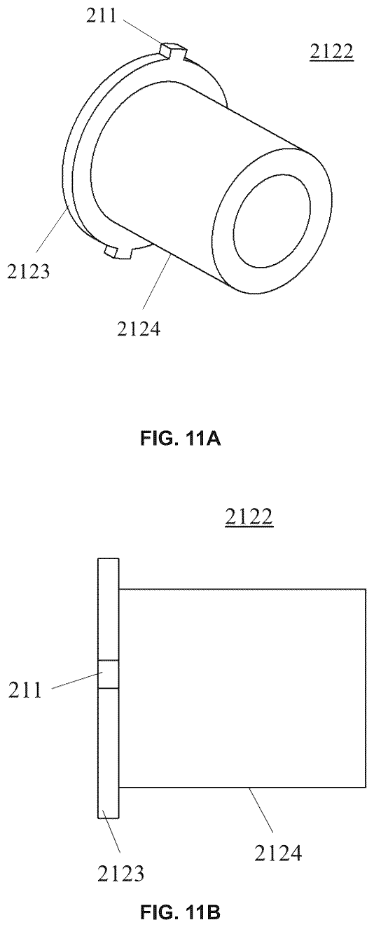

In some embodiments, the sheath 210 includes the elastic member 212 disposed in a passage of the sheath 210. A proximal end of the elastic member 212 is connected to an inner wall of the sheath 210. A distal end of the elastic member 212 is axially movably disposed at the distal end of the sheath 210. The connection projection 2111 is disposed at the distal end of the elastic member 212. In some embodiments, the elastic member 212 includes the spring 2121 and the spring tip 2122. The spring tip 2122 is fixed to a distal end of the spring 2121. The connection projection 2111 is disposed on the spring tip 2122. The spring tip 2122 engages with the connection slot 414 through the connection projection 2111. In some other embodiments, the elastic member 212 is also in other forms, such as a rubber ring, etc. More embodiments regarding the elastic member 212 may be referred to in FIGS. 10A-11B and related descriptions thereof.

In some embodiments, when the connection projection 2111 engages with the connection slot 414, the elastic member 212 is in a stretched state. For example, the elastic member 212 is in the stretched state. The elastic member 212 applies a pulling force toward the proximal direction to the connection projection 2111. As the connection projection 2111 and the connection slot 414 form a limiting engagement, before the accommodating tube 400 and the sheath 210 are released, the elastic member 212 remains in the stretched state.

In some embodiments, when the connection projection 2111 disengages from the connection slot 414, the elastic member 212 drives the connection projection 2111 to separate from the connection slot 414, and the accommodating tube 400 and the sheath 210 are released. In some embodiments, when the sliding portion 140 engages with the first slot 413, the accommodating tube 400 deforms, and the size of the second slot 415 in the first direction becomes greater than or equal to the size of the connection projection 2111 in the first direction. Then, the connection projection 2111 is no longer restrained by the second slot 415. Under an action of its own elastic restoring force, the elastic member 212 pulls the connection projection 2111 to move toward the proximal direction, causing the connection projection 2111 to disengage from the connection slot 414, and the accommodating tube 400 and the sheath 210 are released. By providing the elastic member 212 to drive the connection projection 2111 to quickly separate from the connection slot 414, the efficiency and the reliability of releasing the accommodating tube 400 and the sheath 210 are improved. More exemplary embodiments regarding the connection and the release of the accommodating tube 400 and the sheath 210 may be referred to in FIGS. 3A-4B and the related descriptions thereof.

In some embodiments, the clip arm 100 includes at least two clip portions 120. Each of the at least two clip portions 120 includes the distal joint portion 121, the bending portion 122, and the proximal joint portion 123. The proximal joint portions 123 of the at least two clip portions 120 are integrally formed, thereby improving the stability and the symmetry of the at least two clip portions 120, and improving the clamping stability. More exemplary embodiments regarding the clip arm 100 may be referred to in FIGS. 5A-6B and the related descriptions thereof.

In some embodiments, the proximal joint portion 123 includes the first connection portion 125. The first connection portion 125 includes the pin hole 1251. The clip arm 100 includes the connection pin 130. The connection pin 130 is connected to the pin hole 1251. The connection pin 130 improves the stability and the strength of the proximal joint portion 123. In some embodiments, two end portions of the connection pin 130 constitute the sliding portion 140. The accommodating tube 400 includes two slots 410. The sliding portions 140 at the two ends of the connection pin 130 respectively engage in the two slots 410.

In some embodiments, the proximal joint portion 123 includes the second connection portion 126. The second connection portion 126 includes the connection hole 1261. The clip instrument 10 includes the mandrel 220. A distal end of the mandrel 220 is provided with the connection tip 221. The connection tip 221 is releasably connected to the connection hole 1261. In some embodiments, the connection tip 221 includes the guiding inclined surface 222 and the limiting concession slot 223 located at a proximal end of the guiding inclined surface 222. The guiding inclined surface 222 is configured to guide the connection tip 221 into the connection hole 1261. The connection hole 1261 engages with the limiting concession slot 223, thereby enabling the connection tip 221 to be releasably connected to the clip arm 100.

In some embodiments, the proximal joint portion 123 further includes the installation slot 127. The installation slot 127 extends from the connection hole 1261 to the first connection portion 125. By providing the installation slot 127, when the connection tip 221 is assembled with or disassembled from the proximal joint portion 123, the proximal joint portion 123 elastically deforms and expands outward from the installation slot 127, allowing the connection tip 221 to enter or retract from the connection hole 1261. After the assembly or disassembly is completed, the proximal joint portion 123 returns to its original shape.

With reference to FIGS. 4A-5B, in some embodiments, the accommodating tube 400 includes a second limiting portion 450, and the second limiting portion 450 is disposed at the distal end of the accommodating tube 400. The clip arm 100 includes a stop portion 150, and the stop portion 150 is disposed between the distal joint portion 121 and the bending portion 122. In some embodiments, the second limiting portion 450 includes a stop sheet, and the stop sheet is disposed at the distal end of the accommodating tube 400. In some embodiments, a width of the distal joint portion 121 of the clip arm 100 is greater than a width of the bending portion 122, and a step is formed at a connection between the distal joint portion 121 and the bending portion 122. The step constitutes the stop portion 150.

In some embodiments, after the stop portion 150 abuts against and is limited by the second limiting portion 450, the mandrel 220 moves from the distal direction toward the proximal direction. The mandrel 220 drives the connection tip 221 to disengage from the connection hole 1261, and the clip arm 100 is released from the mandrel 220. For example, when the clip arm 100 moves from the distal direction toward the proximal direction, the step abuts against the stop sheet, and the stop sheet limits the clip arm 100 from continuing to move toward the proximal direction. The mandrel 220 continues to move from the distal direction toward the proximal direction, causing the connection tip 221 to disengage from the connection hole 1261, and the clip arm 100 is released from the mandrel 220.

FIG. 10A is a sectional diagram illustrating an exemplary structure of a clip instrument 10 according to some embodiments of the present disclosure. FIG. 10B is a partial enlarged diagram illustrating a region D of the clip instrument 10 shown in FIG. 10A.

Embodiment Three of the present disclosure provides the clip instrument 10. The clip instrument 10 includes the accommodating tube 400 and the sheath 210.

In some embodiments, the sheath 210 includes the elastic member 212 disposed in a passage of the sheath 210, and the elastic member 212 includes the first connection structure 211. In some embodiments, the elastic member 212 is configured to provide an elastic force along an axial direction of the sheath 210. For example, a proximal end of the elastic member 212 is fixed to an inner wall of the sheath 210, a distal end of the elastic member 212 is provided with the first connection structure 211, and the elastic member 212 generates an elastic force along an axial direction of the sheath 210 through deformation. The elastic force drives the first connection structure 211 to move relative to the sheath 210 in the axial direction.

In some embodiments, the accommodating tube 400 includes the second connection structure 420. The second connection structure 420 and the first connection structure 211 are releasably connected, and the accommodating tube 400 and the sheath 210 are releasably connected. When the first connection structure 211 and the second connection structure 420 are in a limiting fit, the elastic member 212 is in a stretched state, and the elastic member 212 applies a pulling force toward the proximal end to the first connection structure 211. Under the limiting effect of the second connection structure 420 on the first connection structure 211, the elastic member 212 remains in the stretched state. When the limiting between the first connection structure 211 and the second connection structure 420 is released, the elastic member 212 drives the first connection structure 211 to retract from the second connection structure 420, and the accommodating tube 400 is released from the sheath 210. By providing the elastic member 212 to drive the first connection structure 211 to quickly separate from the second connection structure 420, the efficiency and the reliability of releasing the accommodating tube 400 and the sheath 210 are improved.

More exemplary embodiments of the clip instrument 10 in Embodiment Three will be described below with reference to FIGS. 10A-16. It should be understood that some features, structures, or characteristics in Embodiment Three refer to corresponding features, structures, or characteristics in Embodiment One or Embodiment Two, and the present disclosure will not repeat descriptions of some identical features. It should be noted that descriptions of some features in Embodiment Three may refer to FIGS. 1A-9 and the related descriptions.

FIG. 11A is an exemplary structural diagram illustrating the spring tip 2122 according to some embodiments of the present disclosure. FIG. 11B is an exemplary front view of the spring tip 2122 according to some embodiments of the present disclosure.

As shown in FIGS. 10A-11B, in some embodiments, the elastic member 212 includes the spring 2121 and the spring tip 2122. A proximal end of the spring 2121 is fixed to an inner wall of the sheath 210, a distal end of the spring 2121 is fixed to the spring tip 2122, and the spring tip 2122 is movably disposed at a distal end of the sheath 210. At least a portion of the spring tip 2122 extends out of the distal end of the sheath 210 for engaging with the second connection structure 420.

In some embodiments, the spring tip 2122 includes a first engagement portion 2123 and a second engagement portion 2124. The first engagement portion 2123 engages with the accommodating tube 400, the second engagement portion 2124 engages with the sheath 210, and the first connection structure 211 is disposed on the first engagement portion 2123. In some embodiments, the first engagement portion 2123 extends out of the distal end of the sheath 210 and engages with an inner wall of the accommodating tube 400. The first connection structure 211 projects radially outward from a side wall of the first engagement portion 2123 and engages with the second connection structure 420 of the accommodating tube 400.

In some embodiments, an outer diameter of the first engagement portion 2123 is greater than an inner diameter of the sheath 210. For example, a step is formed between the first engagement portion 2123 and the second engagement portion 2124. When the spring 2121 drives the spring tip 2122 to move from the distal direction toward the proximal direction, the step at the proximal end of the first engagement portion 2123 abuts against an end face at the distal end of the sheath 210, thereby preventing the first engagement portion 2123 from entering a passage of the sheath 210. Thus, at least a portion of the spring tip 2122 is always kept outside the passage of the sheath 210 to facilitate reuse.

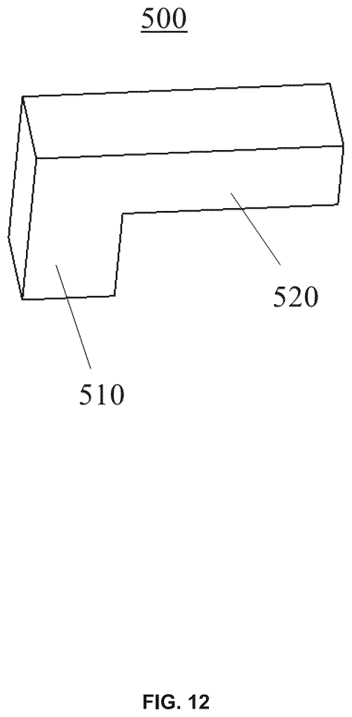

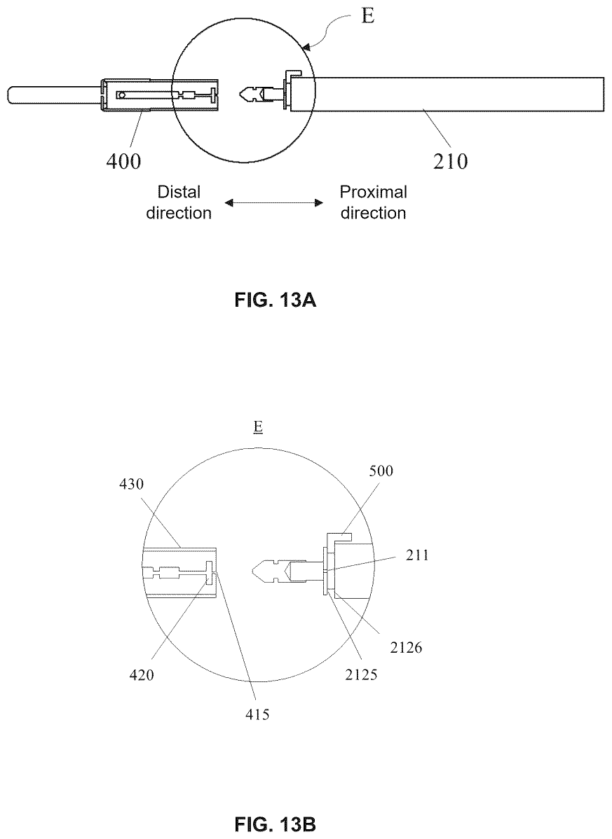

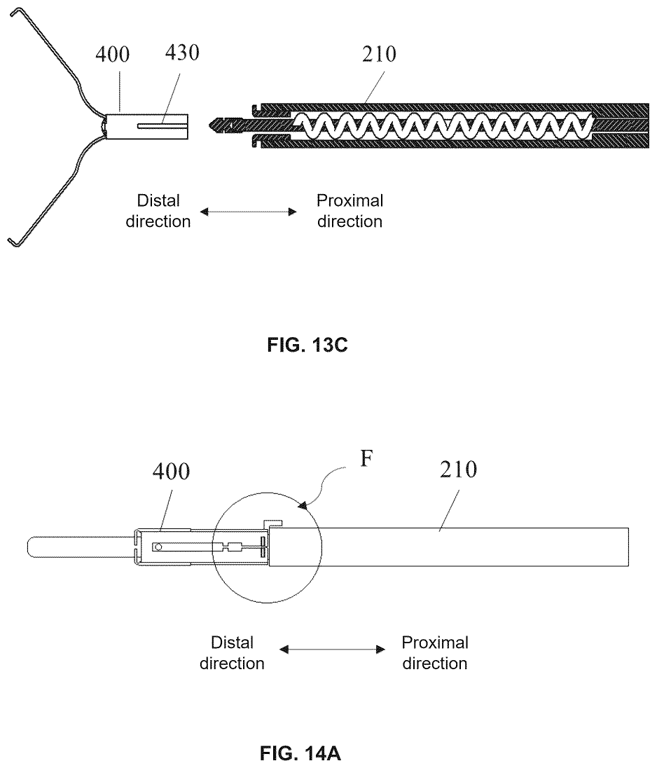

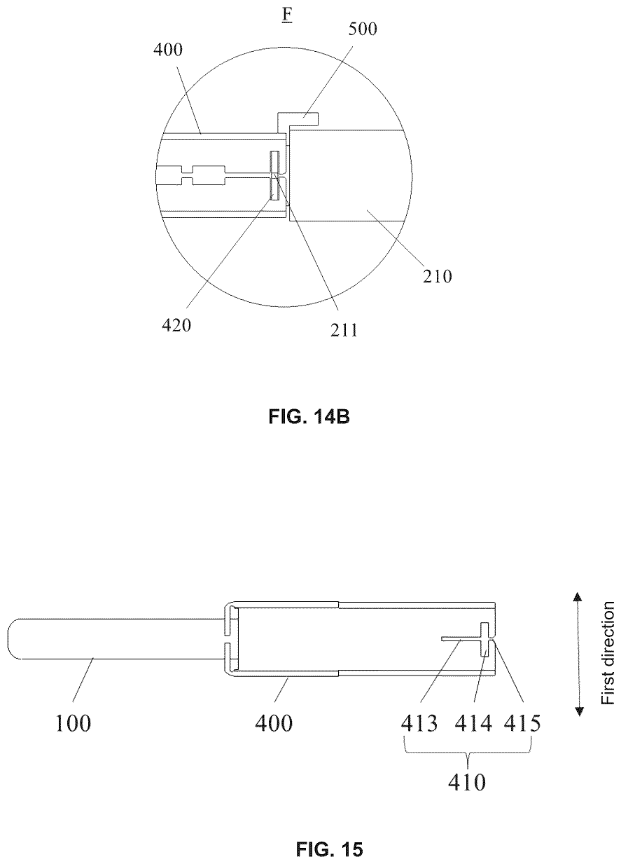

FIG. 12 is an exemplary structural diagram illustrating a limiting member 500 according to some embodiments of the present disclosure. FIGS. 13A-14B are exemplary structural diagrams of assembly of the accommodating tube 400 and the sheath 210 according to some embodiments of the present disclosure.

As shown in FIGS. 12-14B, in some embodiments, the clip instrument 10 further includes the limiting member 500. A proximal end of the first engagement portion 2123 includes a first end face 2125, and a distal end of the sheath 210 includes a second end face 2126. The limiting member 500 is detachably abutted between the first end face 2125 and the second end face 2126, thereby forming an assembly gap between the first connection structure 211 and the second end face 2126. The assembly gap is used for assembling the second connection structure 420. In some embodiments, the limiting member 500 includes an engagement portion 510 and an operation portion 520. The engagement portion 510 is used for being assembled between the first end face 2125 and the second end face 2126. The operation portion 520 projects from the assembly gap and is used for being held by a tool or a hand, thereby facilitating assembly and disassembly.

In some embodiments, a side wall of the accommodating tube 400 includes the concession slot 430. A position of the limiting member 500 along the axial direction of the sheath 210 corresponds to a position of the concession slot 430. The concession slot 430 is used for avoiding the limiting member 500, and enabling a smooth assembly of the accommodating tube 400 and the sheath 210.

In some embodiments, when the accommodating tube 400 and the sheath 210 are in an unconnected state, the limiting member 500 is assembled between the first end face 2125 and the second end face 2126, thereby forming the assembly gap between the first connection structure 211 and the second end face 2126.

In some embodiments, when the sheath 210 moves from the proximal direction toward the distal direction, the limiting member 500 moves into the concession slot 430. In some embodiments, the accommodating tube 400 includes the second slot 415. The second slot 415 is used for allowing the first connection structure 211 to pass through and enter the second connection structure 420. When the sheath 210 moves from the proximal direction toward the distal direction, the first engagement portion 2123 moves into the accommodating tube 400, and the first connection structure 211 presses against an entrance of the second slot 415 and passes through the second slot 415. When the sheath 210 continues to move from the proximal direction toward the distal direction, the first connection structure 211 enters the second connection structure 420. In some embodiments, when the sheath 210 continues to move from the proximal direction toward the distal direction until the first connection structure 211 engages with the second connection structure 420, the accommodating tube 400 and the sheath 210 switch to a connected state.

In some embodiments, after the accommodating tube 400 is connected to the sheath 210, the limiting member 500 is separated between the first end face 2125 and the second end face 2126. In some embodiments, the operation portion 520 of the limiting member 500 is subjected to a force, making the engagement portion 510 to retract from the assembly gap.

By providing the limiting member 500, the assembly gap is formed between the first connection structure 211 and the second end face 2126, thereby avoiding a situation where the first connection structure 211 adheres to the second end face 2126 and cannot be assembled, and making the assembly of the accommodating tube 400 and the sheath 210 simpler.

In some embodiments, the first connection structure 211 includes the connection projection 2111, and the second connection structure 420 further includes the connection slot 414. The connection projection 2111 and the connection slot 414 are releasably connected. In some embodiments, the connection projection 2111 is disposed on the spring tip 2122, and the connection projection 2111 projects radially from the first engagement portion 2123. In some embodiments, the connection slot 414 is disposed at a proximal end of the accommodating tube 400. The accommodating tube 400 further includes the second slot 415, and the second slot 415 extends axially from the connection slot 414 to an edge of the accommodating tube 400. The connection projection 2111 enters and retracts from the connection slot 414 from the second slot 415. In some embodiments, the accommodating tube 400 does not include the second slot 415, and the connection projection 2111 enters the connection slot 414 through an elastic deformation caused by pressing.

FIG. 15 is an exemplary structural diagram illustrating the clip arm 100 and the accommodating tube 400 according to some embodiments of the present disclosure.

In some embodiments, the clip instrument 10 further includes the clip arm 100, and a proximal end of the clip arm 100 is provided with the sliding portion 140. The accommodating tube 400 includes the slot 410. The slot 410 includes the first slot 413 and the connection slot 414, and the first slot 413 is disposed at a distal end of the connection slot 414. When the sliding portion 140 engages with the first slot 413, the engagement between the connection slot 414 and the connection projection 2111 is released. In some embodiments, when the sliding portion 140 slides along an inner wall of the accommodating tube 400, the clip portion 120 switches between an open state and a closed state. When the sliding portion 140 slides into the first slot 413, the connection slot 414 is actuated to deform, causing the connection slot 414 and the connection projection 2111 to release the engagement. In some embodiments, a distal end of the first slot 413 is provided with an entrance allowing the sliding portion 140 to enter the first slot 413. The entrance is greater than a sectional size of the sliding portion 140, so that when the sliding portion 140 slides to the entrance, the sliding portion 140 enters the distal end of the first slot 413.

In some embodiments, the slot 410 further includes the second slot 415. The second slot 415 extends axially from a proximal end of the connection slot 414 to a proximal edge of the accommodating tube 400. A size of the first slot 413 in a first direction is less than a size of the sliding portion 140 in the first direction. When the sliding portion 140 is located in the first slot 413, the accommodating tube 400 deforms such that a size of the second slot 415 in the first direction is greater than or equal to a size of the connection projection 2111 in the first direction, and the connection projection 2111 and the connection slot 414 release engagement. When the sliding portion 140 is not located in the first slot 413, the size of the second slot 415 in the first direction is less than the size of the connection projection 2111 in the first direction, and the proximal end of the connection slot 414 is used to limit the movement of the connection projection 2111 toward the proximal direction. The first direction refers to a direction indicated by the arrow in FIG. 15, or the first direction is understood as a circumferential direction of the accommodating tube 400.

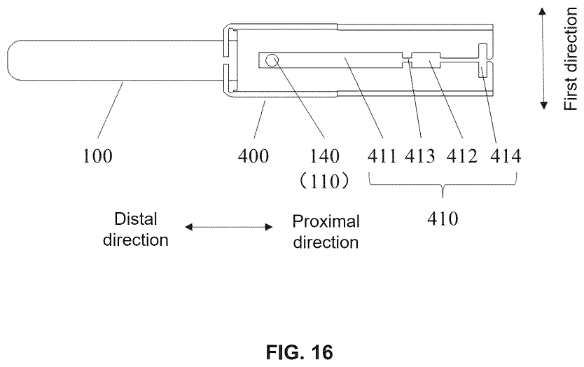

FIG. 16 is an exemplary structural diagram illustrating the clip arm 100 and the accommodating tube 400 according to some other embodiments of the present disclosure.

In some embodiments, the sliding portion 140 constitutes the locking portion 110 of the clip arm 100. The slot 410 further includes the sliding slot 411 and the locking slot 412. The sliding slot 411 is located at a distal direction of the locking slot 412, and the locking slot 412 is located at a distal direction of the connection slot 414. When the locking portion 110 engages with the sliding slot 411, the clip instrument 10 switches between the open state and the closed state. When the locking portion 110 engages with the locking slot 412, the clip instrument 10 is in a locked state. Releasing and locking of the clip arm 100 may be achieved through a simple slot 410. The operation is simple. The slot 410 may maintain the locking reliability and make the release of the accommodating tube 400 more convenient.

In some embodiments, the first slot 413 is located between the sliding slot 411 and the locking slot 412. When the clip arm 100 moves from the distal direction toward the proximal direction, the accommodating tube 400 and the sheath 210 are released first, and then the clip arm 100 is locked with the accommodating tube 400. The sliding portion 140 of the clip arm 100 first actuates the release of the accommodating tube 400 and the sheath 210. Then, the sliding portion 140 engages with the locking slot 412 to achieve locking. After locking, the clip arm 100 is stationary relative to the accommodating tube 400, and the clip arm 100 does not need to continue moving. The reliability and stability of the locking are improved.

In some embodiments, the accommodating tube 400 is rotatably disposed at the distal direction of the sheath 210 around an axis of the sheath 210. This facilitates alignment of a closing direction of the clip portion 120 with a closing direction of a wound. More embodiments regarding the release of the accommodating tube 400 and the sheath 210 may be found in FIGS. 3A-4B and the related descriptions.

In some embodiments, the clip arm 100 includes at least two clip portions 120. Each of the at least two clip portions 120 includes the distal joint portion 121, the bending portion 122, and the proximal joint portion 123. The proximal joint portions 123 of the at least two clip portions 120 are integrally formed.

In some embodiments, the proximal joint portion 123 includes the first connection portion 125. The first connection portion 125 includes the pin hole 1251. The clip arm 100 includes the connection pin 130. The connection pin 130 is connected to the pin hole 1251. Two end portions of the connection pin 130 constitute the sliding portion 140. The proximal joint portion 123 includes the second connection portion 126. The second connection portion 126 includes the connection hole 1261. The clip instrument 10 includes the mandrel 220. The connection tip 221 is provided at a distal end of the mandrel 220. The connection tip 221 is releasably connected to the connection hole 1261.

In some embodiments, the proximal joint portion 123 further includes the installation slot 127. The installation slot 127 extends from the connection hole 1261 to the first connection portion 125.

In some embodiments, the accommodating tube 400 includes a limiting portion. The clip arm 100 includes an abutting portion. After the limiting portion abuts the abutting portion, the mandrel 220 moves from the distal direction toward the proximal direction, and the connection tip 221 disengages from the connection hole 1261. More embodiments regarding the release of the accommodating tube 400 and the sheath 210 may be found in FIGS. 5A-6B and the related descriptions.

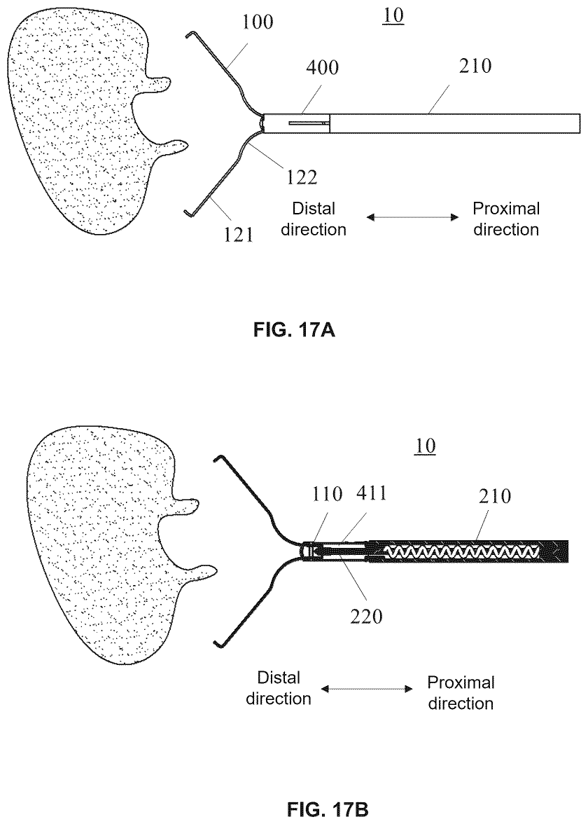

FIGS. 17A-21D are structural diagrams illustrating an exemplary control process of the clip instrument 10 according to some embodiments of the present disclosure.

As shown in FIGS. 17A-17B, the clip arm 100 is in an open state. In some embodiments, the control portion 300 controls the mandrel 220 to move from a proximal direction toward a distal direction, drives the clip arm 100 to move from the proximal direction toward the distal direction. At this time, the locking portion 110 of the clip arm 100 moves from a proximal direction toward a distal direction along the sliding slot 411.

After the bending portion 122 of the clip portion 120 extends out of the accommodating tube 400, the distal joint portion 121 of the clip arm 100 opens under an action of the bending portion 122. A clamping space is formed between the clip portions 120. The control portion 300 controls a movement of the sheath 210 to adjust a position of the clamping space, to align the clamping space with a tissue. The control portion 300 also controls a rotation of the mandrel 220. The mandrel 220 drives the clip arm 100 and the accommodating tube 400 to rotate relative to the sheath 210, to adjust a closing direction of the at least two clip portions 120 to be consistent with a closing direction of a target object.

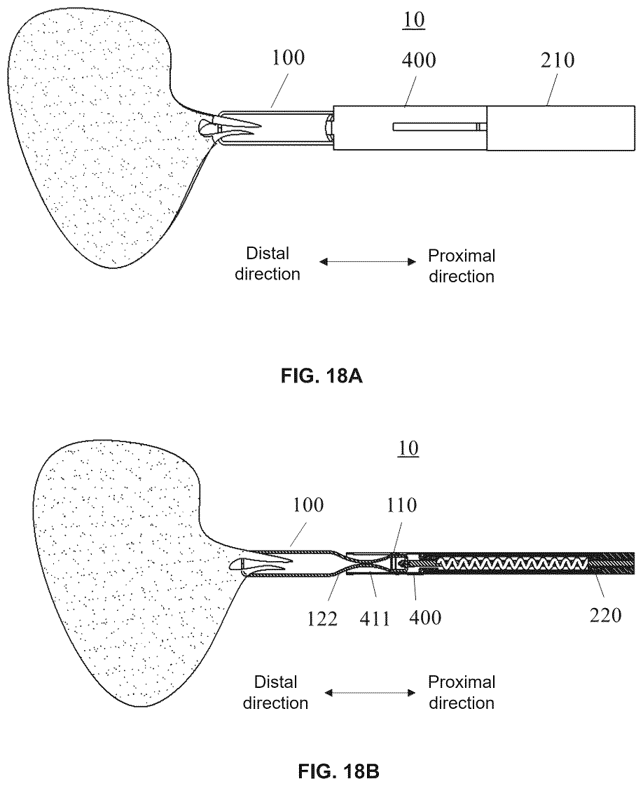

As shown in FIGS. 18A-18B, the clip arm 100 is in a closed state. In some embodiments, the control portion 300 controls the mandrel 220 to move from a distal direction toward a proximal direction, drives the clip arm 100 to move from the distal direction toward the proximal direction. At this time, the locking portion 110 of the clip arm 100 moves from a distal direction toward a proximal direction along the sliding slot 411. After the bending portion 122 of the clip portion 120 is retracted into the accommodating tube 400, the distal joint portion 121 of the clip arm 100 closes under a limiting action of the accommodating tube 400. The at least two clip portions 120 clamp a tissue, causing the tissue to close.

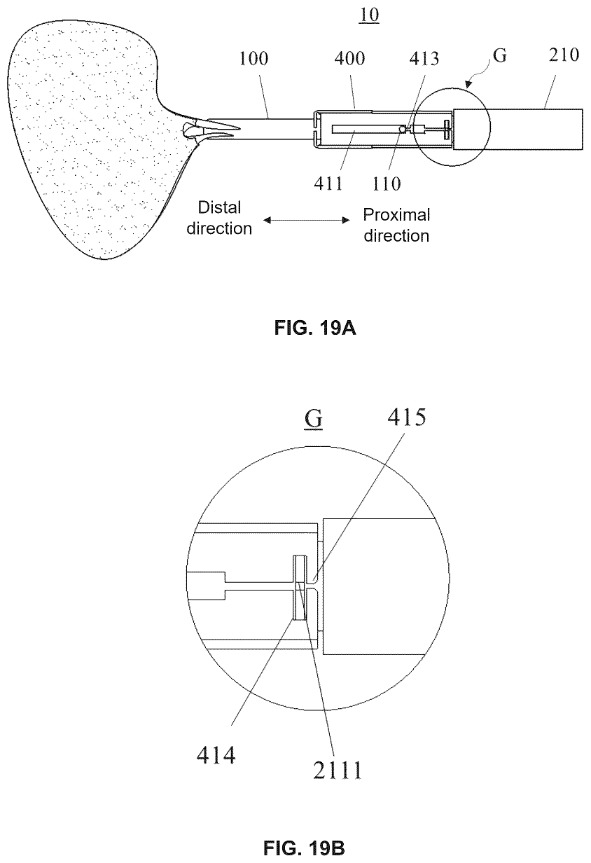

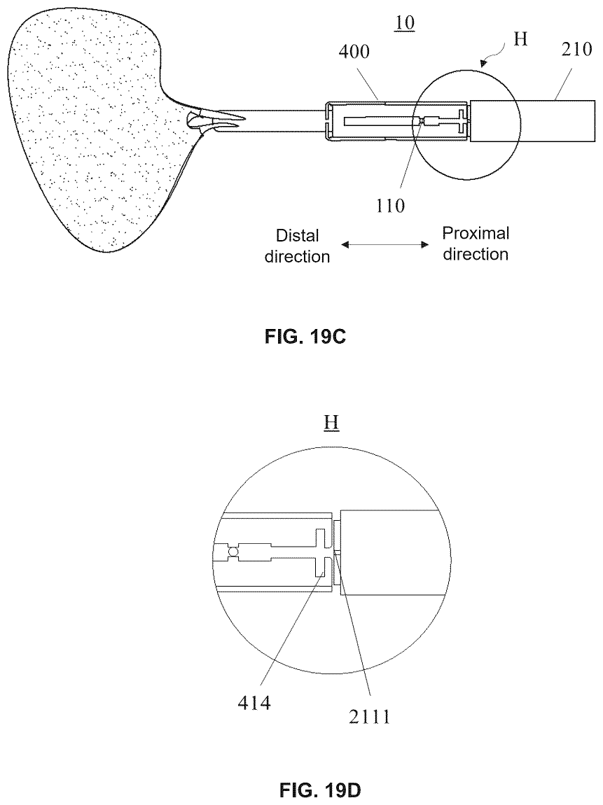

As shown in FIGS. 19A-19D, the accommodating tube 400 and the sheath 210 are released. In some embodiments, the control portion 300 controls the mandrel 220 to move from the distal direction toward the proximal direction, driving the clip arm 100 to move from the distal direction toward the proximal direction. At this time, the locking portion 110 of the clip arm 100 enters the first slot 413 from the sliding slot 411. After the locking portion 110 enters the first slot 413, the locking portion 110 actuates an elastic deformation of a side wall of the accommodating tube 400. The elastic deformation causes a size of the second slot 415 in the first direction to become greater than a size of the connection projection 2111 in the first direction. At this time, an engagement relationship between the connection slot 414 and the connection projection 2111 is released. Under an elastic restoring force, the elastic member 212 drives the spring tip 2122 to move from the distal direction toward the proximal direction, causing the connection projection 2111 to retract from the connection slot 414. The accommodating tube 400 and the sheath 210 are released.

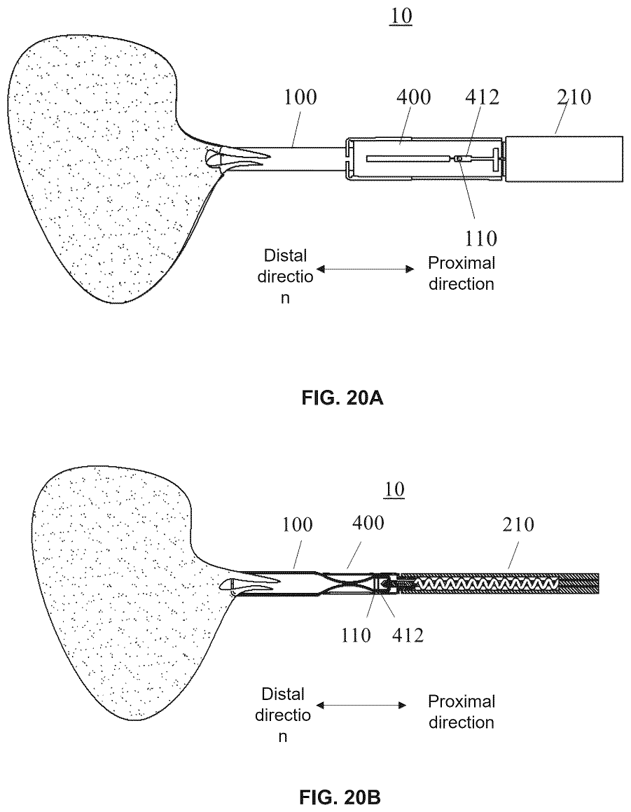

As shown in FIGS. 20A-20B, the clip arm 100 and the accommodating tube 400 are locked. In some embodiments, the control portion 300 controls the mandrel 220 to move from the distal end toward the proximal direction, drives the clip arm 100 to move from the distal end toward the proximal direction. At this time, the locking portion 110 of the clip arm 100 enters the locking slot 412 from the first slot 413, and the accommodating tube 400 returns to an original shape. When the locking portion 110 engages with the locking slot 412, the locking portion 110 limits an axial movement and a circumferential movement of the clip arm 100 relative to the accommodating tube 400, thereby locking the clip arm 100 and the accommodating tube 400.

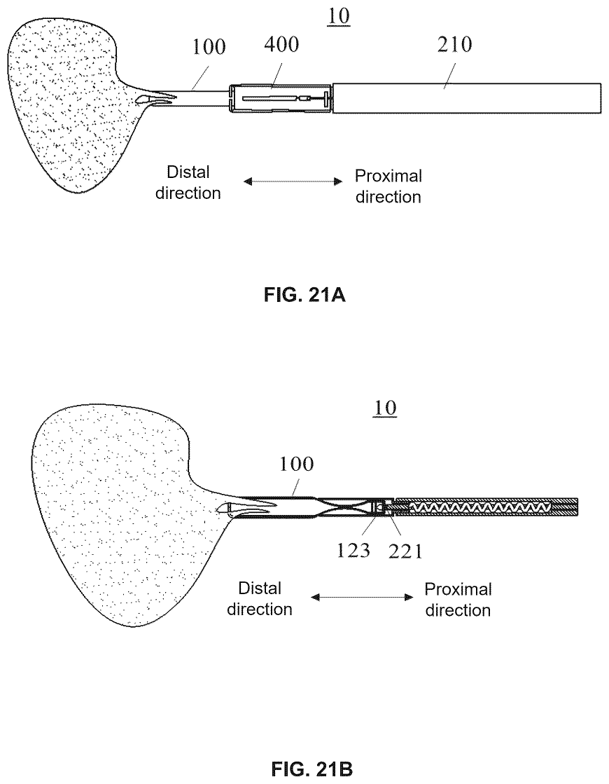

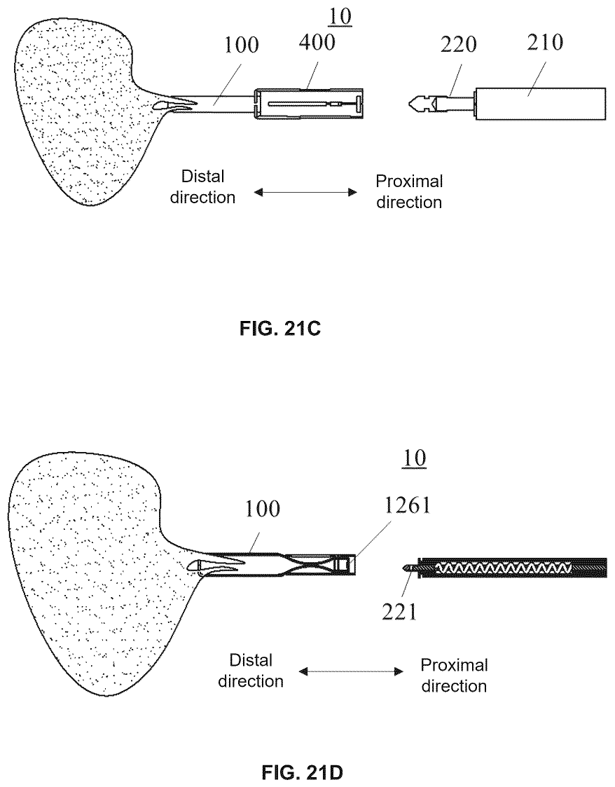

As shown in FIGS. 21A-21D, the mandrel 220 and the clip arm 100 are released. In some embodiments, the control portion 300 controls the mandrel 220 to move from the distal direction toward the proximal direction. At this time, the locking portion 110 of the clip arm 100 is limited within the locking slot 412. A proximal edge of the locking slot 412 limits the movement of the clip arm 100 toward the proximal direction, and/or, after the limiting portion abuts the abutting portion, the limiting portion limits the movement of the clip arm 100 toward the proximal direction. When a driving force value applied by the mandrel 220 to the clip arm 100 increases to a certain degree, the connection tip 221 of the mandrel 220 disengages from the connection hole 1261 of the clip arm 100. The mandrel 220 and the clip arm 100 are released.

Embodiment four of the present disclosure also provides a control method of the clip instrument 10. The control method is applied to the clip instrument 10 in any of the above embodiments.

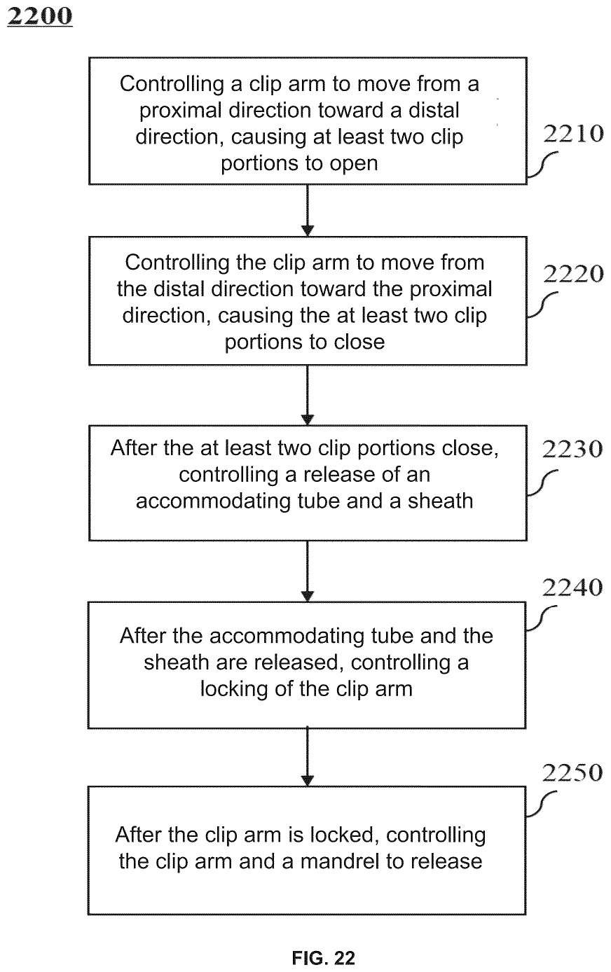

FIG. 22 is an exemplary flowchart illustrating the clip instrument 10 according to some embodiments of the present disclosure. As shown in FIG. 22, a process 2200 includes the following operations. In some embodiments, the process 2200 is executed by the control portion 300. In some embodiments, the clip instrument 10 includes the clip arm 100, the accommodating tube 400, and the sheath 210. The mandrel 220 is disposed inside the sheath 210. The clip arm 100 includes at least two clip portions 120. The accommodating tube 400 is releasably connected to the sheath 210. The clip arm 100 is releasably connected to the mandrel 220.

In operation 2210, the control portion 300 controls the clip arm 100 to move from a proximal direction toward a distal direction, causing the at least two clip portions 120 to open.

In some embodiments, the control portion 300 includes the sliding handle 320. When the sliding handle 320 moves from the proximal direction toward the distal direction, the sliding handle 320 drives the mandrel 220 to move from the proximal direction toward the distal direction, thereby controlling the clip arm 100 to move from the proximal direction toward the distal direction and causing the at least two clip portions 120 to open. In some embodiments, the accommodating tube 400 includes the slot 410. The slot 410 includes the sliding slot 411. The clip arm 100 includes the locking portion 110. The control portion 300 controls, through the mandrel 220, the locking portion 110 to move from the proximal direction toward the distal direction within the sliding slot 411. After the bending portion 122 of the clip portion 120 extends out of the accommodating tube 400, the distal joint portions 121 move away from each other under an action of the bending portion 122, causing the at least two clip portions 120 to open.

In operation 2220, the control portion 300 controls the clip arm 100 to move from the distal direction toward the proximal direction, causing the at least two clip portions 120 to close.

In some embodiments, the control portion 300 drives the mandrel 220 to move from the distal direction toward the proximal direction, thereby controlling the clip arm 100 to move from the distal direction toward the proximal direction and cause the at least two clip portions 120 to close. In some embodiments, the control portion 300 controls, through the mandrel 220, the locking portion 110 to move from the distal end toward the proximal end within the sliding slot 411. After the bending portion 122 of the clip portion 120 is retracted into the accommodating tube 400, the bending portion 122, under a limiting action of the accommodating tube 400, drives the distal joint portions 121 to move toward each other, causing the at least two clip portions 120 to close.

In operation 2230, after the at least two clip portions 120 close, the control portion 300 controls the release of the accommodating tube 400 and the sheath 210.

In some embodiments, the slot 410 includes the connection slot 414 and the first slot 413. The sheath 210 includes the elastic member 212. The elastic member 212 includes the connection projection 2111. The connection slot 414 is releasably connected to the connection projection 2111.

In some embodiments, the control portion 300 drives the mandrel 220 to move from the distal direction toward the proximal direction, thereby controlling the clip arm 100 to move from the distal direction toward the proximal direction. The locking portion 110 of the clip arm 100 engages with the first slot 413, causing the release of the connection slot 414 and the connection projection 2111. In some embodiments, the control portion 300 controls the locking portion 110 to engage with the first slot 413, to actuate a displacement of the connection slot 414. The displacement is caused by an elastic deformation of the accommodating tube 400. After the connection slot 414 is displaced, the connection projection 2111 and the connection slot 414 are disengaged from a limiting relationship.

In some embodiments, after the connection projection 2111 and the connection slot 414 are disengaged from the limiting relationship, the connection projection 2111 is controlled to retract from the connection slot 414. The accommodating tube 400 and the sheath 210 are released. In some embodiments, the sheath 210 further includes an elastic member 212. When the connection projection 2111 engages with the connection slot 414, the elastic member 212 is in a stretched state. When the connection projection 2111 and the connection slot 414 are disengaged from engagement, the elastic member 212, under an elastic restoring force, drives the spring tip 2122 to move from the distal direction toward the proximal direction, causing the connection projection 2111 to retract from the connection slot 414. The accommodating tube 400 and the sheath 210 are released.

In operation 2240, after the accommodating tube 400 and the sheath 210 are released, the control portion 300 controls a locking of the clip arm 100.

In some embodiments, the slot 410 of the accommodating tube 400 includes the locking slot 412. The locking slot 412 is located at the proximal direction of an actuation structure. In some embodiments, the control portion 300 drives the mandrel 220 to move from the distal direction toward the proximal direction, thereby controlling the clip arm 100 to move from the distal direction toward the proximal direction. The clip arm 100 controls the locking portion 110 to move from the distal direction toward the proximal direction, causing the locking portion 110 to engage with the locking slot 412, and the at least two clip portions 120 are locked.

When the locking portion 110 engages with the locking slot 412, the locking portion 110 may limit an axial movement and a circumferential movement of the clip arm 100 relative to the accommodating tube 400, thereby improving the reliability of locking. The sliding portion 140 of the clip arm 100 first actuates the accommodating tube 400 and the sheath 210 to release, and then the sliding portion 140 engages with the locking slot 412 to achieve locking. Then, after locking, the clip arm 100 is stationary relative to the accommodating tube 400, and the clip arm 100 does not need to continue moving, thereby improving the reliability and the stability of locking.

In operation 2250, after the clip arm 100 is locked, the control portion 300 controls the clip arm 100 and the mandrel 220 to release.

In some embodiments, the accommodating tube 400 includes a limiting portion. The limiting portion is disposed at the proximal direction of the locking slot 412. The clip arm 100 includes the stop portion 150. The locking portion 110 of the clip arm 100 is configured as the stop portion 150. In some embodiments, the accommodating tube 400 includes the limiting portion. The limiting portion is disposed at the distal end of the accommodating tube 400. The clip arm 100 includes the stop portion 150. A step is formed between the distal joint portion 121 and the bending portion 122 of the clip arm 100. The step is configured as the stop portion 150.

In some embodiments, the control portion 300 drives the mandrel 220 to move from the distal direction toward the proximal direction, thereby controlling the clip arm 100 to move from the distal direction toward the proximal direction, and thereby controlling the stop portion 150 to move from the distal direction toward the proximal direction and abut against the limiting portion. The limiting portion is configured to prevent the locking portion 110 from continuing to move toward the proximal direction. In some embodiments, the control portion 300 controls the mandrel 220 to move from the distal direction toward the proximal direction. When a driving force value applied by the mandrel 220 to the clip arm 100 increases to a certain extent, the connection tip 221 of the mandrel 220 and the at least two clip portions 120 are released.

It should be noted that the above description of the process 2200 is merely for illustration and explanation, and does not limit the scope of application of the present disclosure. For those skilled in the art, various modifications, and changes to the process 2200 may be made under the guidance of the present disclosure. However, these modifications and changes are still within the scope of the present disclosure. For example, the locking slot is changed to another form of locking member, the sliding slot of the accommodating tube is canceled, and the locking portion 110 (also referred to as the sliding portion) of the clip arm slides directly on an inner wall of the accommodating tube, etc.

Beneficial effects that may be brought by embodiments of the present application include but are not limited to the following contents.

(1) The side wall of the accommodating tube includes the slot. The sliding slot and the locking slot are both concentrated in the slot, thereby simplifying an overall structure. By providing the slot, the release process and the locking process of the clip arm can be achieved through the simple slot. The operation is simple, which maintains the reliability of locking and makes the release of the accommodating tube more convenient.

(2) The locking portion of the clip arm can directly enter the locking slot by moving from the sliding slot toward the proximal direction, making the locking operation more convenient. Furthermore, on the basis that the accommodating tube itself has a locking effect on the clip arm, the locking portion is locked again through the locking slot. The locking portion can limit a movement of the locking portion within a certain range. The locking portion limits the clip arm from moving toward the distal direction to prevent the clip portion from opening again. The locking portion also limits the clip arm from moving toward the proximal direction to prevent the clip portion from excessively entering the accommodating tube. The locking portion also limits the clip arm from moving in a circumferential direction relative to the accommodating tube to prevent relative rotation between the clip arm and the accommodating tube. Therefore, the reliability of locking between the clip arm and the accommodating tube is improved.

(3) During a process in which the clip arm moves from the distal direction toward the proximal direction, the accommodating tube and the sheath are released first. Then, the clip arm is locked with the accommodating tube. This sequence ensures that the clip arm no longer continues to move after locking, guaranteeing the reliability of locking.

(4) The locking portion actuates the first slot, causing the side wall of the accommodating tube to undergo elastic deformation. The elastic deformation causes a size of the first slot in the first direction to increase, thereby disengaging an engagement relationship between the connection projection and the connection slot. This improves the convenience of releasing the accommodating tube and the sheath.

(5) By providing the concession slot, the side wall of the accommodating tube deforms more easily, avoiding excessive resistance when the locking portion moves within the first slot.

(6) The proximal joint portions of the at least two clip portions are integrally formed, thereby improving the stability and the symmetry of the at least two clip portions, and improving the stability of clipping.

(7) By providing the elastic member to drive the first connection structure to quickly separate from the second connection structure, the efficiency and the reliability of releasing the accommodating tube and the sheath are improved.

(8) By providing the limiting member, the assembly gap is formed between the first connection structure and the second end face. This avoids a situation where the first connection structure adheres to the second end face and cannot be assembled, making the assembly of the accommodating tube and the sheath simpler.

It should be noted that different embodiments may produce different beneficial effects. In different embodiments, the beneficial effects produced may be any one or a combination of the above, or may be any other beneficial effects that may be obtained.

The basic concepts have been described above. Obviously, for those skilled in the art, the above detailed disclosure is merely an example and does not constitute a limitation on the present disclosure. Although not explicitly stated herein, those skilled in the art may make various modifications, improvements, and corrections to the present disclosure. Such modifications, improvements, and corrections are suggested in the present disclosure, so such modifications, improvements, and corrections still fall within the spirit and scope of the exemplary embodiments of the present disclosure.

Furthermore, unless explicitly stated in the claims, the order of processing elements and sequences, the use of numbers and letters, or the use of other names in the present disclosure are not intended to limit the order of the processes and methods of the present disclosure. Although the foregoing disclosure discusses some embodiments currently considered useful through various examples, it should be understood that such details are for illustrative purposes only. The appended claims are not limited to the disclosed embodiments. On the contrary, the claims are intended to cover all modifications and equivalent combinations that conform to the essence and scope of the embodiments of the present disclosure.

Similarly, it should be noted that, in order to simplify the expression disclosed in the present disclosure and thereby help understand one or more embodiments, various features are sometimes grouped into one embodiment, drawing, or description thereof in the foregoing description of the embodiments of the present disclosure. However, this disclosure manner does not mean that the object of the present disclosure requires more features than those mentioned in the claims. Rather, claimed subject matter may lie in less than all features of a single foregoing disclosed embodiment.

In some embodiments, numbers describing quantities of components and attributes are used. It should be understood that such numbers used to describe the embodiments are modified by the modifiers “about,” “approximately,” or “substantially” in some examples. Unless otherwise stated, “about,” “approximately,” or “substantially” indicates that the stated number allows a variation of +20%. Accordingly, in some embodiments, the numerical parameters used in the present disclosure and claims are approximate values. The approximate values may vary according to the characteristics required by individual embodiments. In some embodiments, the numerical parameters should consider the prescribed number of significant digits and adopt a general method of digit retention. Although the numerical ranges and parameters used to confirm the breadth of the scope in some embodiments of the present disclosure are approximate values, in specific embodiments, the setting of such numerical values is as precise as possible within a feasible range.

Finally, it should be understood that the embodiments described in the present disclosure are only used to illustrate the principles of the embodiments of the present disclosure. Other variations may also fall within the scope of the present disclosure. Therefore, by way of example and not limitation, alternative configurations of the embodiments of the present disclosure may be considered consistent with the teachings of the present disclosure. Accordingly, the embodiments of the present disclosure are not limited to the embodiments explicitly introduced and described in the present disclosure.

What is claimed is:

1. A clip instrument, comprising:
a clip arm, a proximal end of the clip arm being provided with a locking portion;
an accommodating tube, the proximal end of the clip arm being movably disposed in the accommodating tube, the accommodating tube including at least one slot extending along an axial direction, and the at least one slot including a sliding slot, a locking slot, and a first slot; the sliding slot being located in a distal direction of the locking slot, and the first slot being located between the sliding slot and the locking slot;
wherein when the locking portion is engaged in the sliding slot, the clip arm switches between an open state and a closed state; and
when the locking portion passes over the first slot and is engaged in the locking slot, the clip arm is in a locked state.

2. The clip instrument of claim 1, further comprising a sheath, wherein
the accommodating tube is releasably connected to the sheath; and
when the locking portion is located in the first slot, the accommodating tube and the sheath are released.

3. The clip instrument of claim 2, wherein the sheath includes a first connection structure, the accommodating tube includes a second connection structure, and the second connection structure is releasably connected to the first connection structure; and
when the locking portion is located in the first slot, the second connection structure is caused to be displaced, causing disengagement of the first connection structure and the second connection structure.

4. The clip instrument of claim 3, wherein the first connection structure includes a connection lug; and
the at least one slot further includes a connection slot located in a proximal direction of the locking slot, the connection slot constitutes the second connection structure, and the connection lug is releasably connected to the connection slot.

5. The clip instrument of claim 4, wherein the at least one slot further includes a second slot, and the second slot extends from a proximal end of the connection slot along an axial direction of the accommodating tube to a proximal edge of the accommodating tube;
a dimension of the first slot in a first direction is smaller than a dimension of the locking portion in the first direction;
when the locking portion is engaged in the first slot, the accommodating tube deforms such that a dimension of the second slot in the first direction is greater than or equal to a dimension of the connection lug in the first direction, and the connection lug disengages from the connection slot;
when the locking portion is not engaged in the first slot, the dimension of the second slot in the first direction is smaller than the dimension of the connection lug in the first direction, and a proximal end of the connection slot restricts a movement of the connection lug along a proximal direction.

6. The clip instrument of claim 5, wherein the at least one slot further includes a third slot, and the third slot extends from the locking slot to the connection slot; a dimension of the third slot in the first direction is smaller than the dimension of the locking portion in the first direction.

7. The clip instrument of claim 1, wherein the accommodating tube further includes at least one concession slot, the concession slot extends from a middle portion of the accommodating tube to a proximal edge of the accommodating tube, and the concession slot and the at least one slot are spaced apart along a circumferential direction of the accommodating tube.

8. The clip instrument of claim 3, wherein the sheath includes an elastic member disposed in a passage of the sheath, a proximal end of the elastic member is connected to an inner wall of the sheath, a distal end of the elastic member is axially movably disposed at a distal end of the sheath, and the first connection structure is provided at the distal end of the elastic member;
when the second connection structure is engaged with the first connection structure, the elastic member is in a stretched state; and
when the second connection structure is disengaged with the first connection structure, the elastic member drives the second connection structure to separate from the first connection structure, and the accommodating tube and the sheath are released.

9. The clip instrument of claim 2, wherein the accommodating tube is rotatably disposed at a distal direction of the sheath about an axis of the sheath.

10. The clip instrument of claim 1, wherein the clip arm includes at least two clip portions, each of the at least two clip portions includes a distal joint portion, a bending portion, and a proximal joint portion, and the proximal joint portions of the at least two clip portions are integrally formed.

11. The clip instrument of claim 10, wherein the proximal joint portion includes a first connection portion, the first connection portion includes a pin hole, the clip arm includes a connection pin, the connection pin is connected to the pin hole, and two end portions of the connection pin constitute the locking portion; and
the proximal joint portion includes a second connection portion, the second connection portion includes a connection hole, the clip instrument comprises a mandrel, a distal end of the mandrel is provided with a connection tip, and the connection tip is releasably connected to the connection hole.

12. The clip instrument of claim 11, wherein the proximal joint portion further includes an installation slot, and the installation slot extends from the connection hole to the first connection portion.

13. The clip instrument of claim 11, wherein the at least one slot includes a first limiting portion, and the first limiting portion is provided at a proximal direction of the locking slot; and
after the connection pin abuts against and is limited by the first limiting portion, the mandrel moves from a distal direction toward a proximal direction, the mandrel drives the connection tip to disengage from the connection hole, and the clip arm and the mandrel are released.

14. The clip instrument of claim 7, wherein both ends of the concession slot along an axial direction of the concession slot are closed.

15. The clip instrument of claim 7, wherein the concession slot includes an opening facing a proximal direction of the accommodating tube.

16. The clip instrument of claim 7, wherein a slot wall at an end of the first slot connected to the locking slot constitutes a third limiting portion, and the third limiting portion restricts the locking portion from moving from the locking slot toward the distal end direction.

17. The clip instrument of claim 16, wherein a proximal edge of the concession slot is closer to the proximal edge of the accommodating tube than the third limiting portion, and a distal edge of the concession slot is closer to a distal edge of the accommodating tube than the third limiting portion.

18. The clip instrument of claim 1, wherein a dimension of the first slot in a first direction is smaller than a dimension of the locking portion in the first direction; and when the locking portion is located at a proximal end of the sliding slot, a distal end of the first slot forms a stop against the locking portion.

19. The clip instrument of claim 1, wherein the clip arm includes a connection pin, and two end portions of the connection pin constitute the locking portion.

20. A control method of a clip instrument, wherein the clip instrument comprises a clip arm and an accommodating tube, a proximal end of the clip arm is provided with a locking portion; the proximal end of the clip arm is movably disposed in the accommodating tube, the accommodating tube includes at least one slot extending along an axial direction, the at least one slot includes a sliding slot, a locking slot, and a first slot, the sliding slot is located in a distal direction of the locking slot, the first slot is located between the sliding slot and the locking slot, and the first slot is in communication with the sliding slot and the locking slot at two ends, respectively;
the control method comprising:
controlling the locking portion to move from a distal direction toward a proximal direction within the sliding slot, causing the clip arm to switch from an open state to a closed state;
controlling the locking portion to move toward a proximal direction to enter the first slot from the sliding slot, the clip arm maintaining the closed state when the locking portion is located in the first slot; and
controlling the locking portion to move toward the proximal direction to enter the locking slot from the first slot, the clip arm being in a locked state when the locking portion is located in the locking slot.
